(19) United States
(12) Patent Application Publication
(10) Pub. No.: US 2026/0074011 A1
(43) Pub. Date: Mar. 12, 2026

(54) END-TO-END MACHINE LEARNING-DRIVEN DESIGN OF PROTEINS

(71) Applicant: Massachusetts Institute of Technology, Cambridge, MA (US)

(72) Inventors: Lin Li, Lincoln, MA (US); Matthew Edmund Walsh, Baltimore, MD (US); Tristan Bepler, Cambridge, MA (US); Leslie Ka-Yan Shing, Woburn, MA (US); John William Spaeth, Somerville, MA (US); Esther Wolf, Concord, MA (US); Rafael Jaimes, Canton, MA (US); Rajmonda Sulo Caceres, Sudbury, MA (US)

(73) Assignee: Massachusetts Institute of Technology, Cambridge, MA (US)

(21) Appl. No.: 19/106,578

(22) PCT Filed: Aug. 25, 2023
(86) PCT No.: PCT/US2023/031156

§ 371 Date: Feb. 25, 2025

Related U.S. Application Data
(60) Provisional application No. 63/373,682, filed on Aug. 26, 2022.

(51) Int. Cl. G16B 15/30; G16B 5/20; G16B 30/00; G16B 40/20

(52) U.S. Cl. CPC G16B 15/30; G16B 5/20; G16B 30/00; G16B 40/20

(57) ABSTRACT

Described herein are techniques for designing proteins for binding to a target. In some embodiments, the techniques include: obtaining an amino acid sequence for a candidate protein that binds to the target with a candidate binding affinity; determining, for proteins in a set of proteins, probabilities that binding affinities between the proteins and the target are greater than the candidate binding affinity, and identifying a subset of the set of proteins based on the determined probabilities. Determining a first probability that a first binding affinity between a first protein and the target is greater than the candidate binding affinity may include: processing a first amino acid sequence of the first protein using a trained machine learning model to obtain a first output indicative of the first binding affinity; and determining the first probability using the first output indicative of the first binding affinity between the first protein and the target.

### RELATED APPLICATIONS

This application claims priority under 35 U.S.C. § 119 (e) to U.S. provisional application No. 63/373,682, filed Aug. 26, 2022, entitled “END-TO-END MACHINE LEARNING-DRIVEN DESIGN OF TARGETED MONOCLONAL ANTIBODIES,” which application is incorporated by reference in its entirety.

### FEDERALLY SPONSORED

This invention was made with government support under FA8702-15-D-0001 awarded by the U.S. Air Force. The government has certain rights in the invention.

### BACKGROUND

Proteins are molecules composed of one or more linear chains of amino acids (i.e., polypeptides). An antibody is a protein component of the immune system. Antibodies are composed of polypeptide chains including light chains and heavy chains. Regions of the polypeptide chains form antigen-binding fragments (Fab). Fabs are responsible for recognizing and binding to antigens.

### BRIEF SUMMARY

Some aspects provide for a method for designing antibodies for binding to a target, the method comprising: obtaining an amino acid sequence of a candidate antibody wherein the candidate antibody binds to the target with a candidate binding affinity; determining, for antibodies in a set of antibodies, probabilities that binding affinities between the antibodies and the target are greater than the candidate binding affinity, the antibodies in the set of antibodies having different amino acid sequences, the antibodies including a first antibody having a first amino acid sequence, and the probabilities including a first probability that a first binding affinity between the first antibody and the target is greater than the candidate binding affinity, wherein determining the first probability comprises: processing the first amino acid sequence of the first antibody using a trained machine learning model to obtain a first output indicative of the first binding affinity between the first antibody and the target; and determining the first probability using the first output indicative of the first binding affinity between the first antibody and the target; and identifying a subset of the set of antibodies based on the determined probabilities that the binding affinities are greater than the candidate binding affinity.

Some aspects provide for a system, comprising: at least one computer hardware processor; and at least one non-transitory computer-readable storage medium storing processor-executable instructions that, when executed by the at least one computer hardware processor, cause the at least one computer hardware processor to perform a method for designing antibodies for binding to a target, the method comprising: obtaining an amino acid sequence of a candidate antibody wherein the candidate antibody binds to the target with a candidate binding affinity; determining, for antibodies in a set of antibodies, probabilities that binding affinities between the antibodies and the target are greater than the candidate binding affinity, the antibodies in the set of antibodies having different amino acid sequences, the antibodies including a first antibody having a first amino acid sequence, and the probabilities including a first probability that a first binding affinity between the first antibody and the target is greater than the candidate binding affinity, wherein determining the first probability comprises: processing the first amino acid sequence of the first antibody using a trained machine learning model to obtain a first output indicative of the first binding affinity between the first antibody and the target; and determining the first probability using the first output indicative of the first binding affinity between the first antibody and the target; and identifying a subset of the set of antibodies based on the determined probabilities that the binding affinities are greater than the candidate binding affinity.

Some aspects provide for at least one non-transitory computer-readable storage medium storing processor-executable instructions that, when executed by at least one computer hardware processor, cause the at least one computer hardware processor to perform a method for designing antibodies for binding to a target, the method comprising: obtaining an amino acid sequence of a candidate antibody, wherein the candidate antibody binds to the target with a candidate binding affinity; determining, for antibodies in a set of antibodies, probabilities that binding affinities between the antibodies and the target are greater than the candidate binding affinity, the antibodies in the set of antibodies having different amino acid sequences, the antibodies including a first antibody having a first amino acid sequence, and the probabilities including a first probability that a first binding affinity between the first antibody and the target is greater than the candidate binding affinity, wherein determining the first probability comprises: processing the first amino acid sequence of the first antibody using a trained machine learning model to obtain a first output indicative of the first binding affinity between the first antibody and the target; and determining the first probability using the first output indicative of the first binding affinity between the first antibody and the target; and identifying a subset of the set of antibodies based on the determined probabilities that the binding affinities are greater than the candidate binding affinity.

Some embodiments further comprise producing at least one antibody in the identified subset of the set of antibodies.

In some embodiments, determining the probabilities that the binding affinities between the antibodies and the target are greater than the candidate binding affinity further comprises: determining a second amino acid sequence of a second antibody in the set of antibodies based on (i) the first probability that the first binding affinity is greater than the candidate binding affinity and (ii) the first amino acid sequence of the first antibody.

Some embodiments further comprise: after determining the second amino acid sequence, determining a second probability that a second binding affinity between the second antibody and the target is greater than the candidate binding affinity.

In some embodiments, determining the second amino acid sequence of the second antibody comprises performing at least a portion of a sampling algorithm to determine the second amino acid sequence.

In some embodiments, the sampling algorithm is a hill climb algorithm, a genetic algorithm, or a Gibbs sampling algorithm.

Some embodiments further comprise: identifying the first antibody from among a training set of antibodies having known binding affinities.

In some embodiments, identifying the subset of the set of antibodies comprises: ranking the antibodies in the set of antibodies by the probabilities determined for the antibodies; and identifying the subset of the set of antibodies based on the ranking.

In some embodiments, ranking the antibodies in set of antibodies by the probabilities determined for the antibodies comprises ranking the antibodies from a highest probability of the determined probabilities to a lowest probability of the determined probabilities.

In some embodiments, identifying the subset of the set of antibodies based on the ranking comprises identifying a predetermined number of antibodies associated with highest probabilities of the determined probabilities.

Some embodiments further comprise: identifying, from among the identified subset of the set of antibodies, one or more antibodies having at least one pre-determined property; and producing the identified one or more antibodies having the at least one pre-determined property.

In some embodiments, the first output indicative of the first binding affinity between the first antibody and the target comprises a mean of the first binding affinity and a standard deviation of the first binding affinity.

In some embodiments, determining the first probability using the first output indicative of the first binding affinity between the first antibody and the target comprises determining the first probability using the mean of the first binding affinity and the standard deviation of the first binding affinity.

In some embodiments, the trained machine learning model comprises at least one regression model.

In some embodiments, the at least one regression model is trained to predict, for an amino acid sequence of an antibody, a binding affinity between the antibody and the target.

In some embodiments, the at least one regression model comprises multiple regression models, wherein each of the multiple regression models is trained to predict, for an amino acid sequence of an antibody, a binding affinity between the antibody and the target, and an output of the at least one regression model comprises a mean of the binding affinities predicted by the multiple regression models and a standard deviation of the binding affinities predicted by the multiple regression models.

In some embodiments, the trained machine learning model comprises a Gaussian Process model.

In some embodiments, the Gaussian Process model is trained to predict, for an amino acid sequence of an antibody, a binding affinity between the antibody and the target, and the probabilistic model is trained to output a mean of the predicted binding affinity and standard deviation of the predicted binding affinity.

In some embodiments, the trained machine learning model comprises at least one language model trained to encode amino acid sequences.

In some embodiments, the at least one language model is trained to predict masked amino acids in at least one amino acid sequence.

In some embodiments, the trained machine learning model further comprises: at least one regression model fine-tuned from the at least one language model, or a probabilistic model fine-tuned from the at least one language model.

In some embodiments, processing the first amino acid sequence of the first antibody using the trained machine learning model comprises: processing the first amino acid sequence using the at least one language model to obtain an encoded amino acid sequence, and processing the encoded amino acid sequence using the at least one regression model or the at least one probabilistic model to obtain the first output indicative of the first binding affinity between the first antibody and the target.

In some embodiments, the at least one language model is a bidirectional encoder representations from transformers (BERT) model.

In some embodiments, the antibodies in the set of antibodies include single-chain variable fragments (scFvs).

Some aspects provide for a method of training a machine learning model to predict binding affinities between antibodies and a target, the method comprising: using at least one computer hardware processor to perform: training at least one language model to encode amino acid sequences; obtaining training data using a candidate amino acid sequence of a candidate antibody, wherein the candidate antibody binds to the target with a candidate binding affinity; and training the machine learning model to predict the binding affinities between the antibodies and the target using the at least one trained language model and the obtained training data.

Some aspects provide for a system, comprising: at least one computer hardware processor; and at least one non-transitory computer-readable storage medium storing processor-executable instructions that, when executed by the at least one computer hardware processor, cause the at least one computer hardware processor to perform a method of training a machine learning model to predict binding affinities between antibodies and a target the method comprising: training at least one language model to encode amino acid sequences; obtaining training data using a candidate amino acid sequence of a candidate antibody, wherein the candidate antibody binds to the target with a candidate binding affinity; and training the machine learning model to predict the binding affinities between the antibodies and the target using the at least one trained language model and the obtained training data.

Some aspects provide for at least one non-transitory computer-readable storage medium storing processor-executable instructions that, when executed by at least one computer hardware processor, cause the at least one computer hardware processor to perform a method of training a machine learning model to predict binding affinities between antibodies and a target the method comprising: training at least one language model to encode amino acid sequences; obtaining training data using a candidate amino acid sequence of a candidate antibody, wherein the candidate antibody binds to the target with a candidate binding affinity; and training the machine learning model to predict the binding affinities between the antibodies and the target using the at least one trained language model and the obtained training data.

In some embodiments, training the at least one language model to encode the amino acid sequences comprises training the at least one language model to predict masked amino acids in at least one amino acid sequence.

In some embodiments, training the at least one language model comprises training a protein language model using protein training data, the protein training data comprising amino acid sequences for individual protein domains.

In some embodiments, the amino acid sequences for the individual protein domains include at least 1 million amino acid sequences, at least 10 million amino acid sequences, at least 15 million amino acid sequences, at least 20 million amino acid sequences, at least 25 million amino acid sequences, or at least 30 million amino acid sequences.

In some embodiments, training the at least one language model comprises training a heavy chain language model using heavy chain training data, the heavy chain training data comprising amino acid sequences of antibody heavy chains.

In some embodiments, the amino acid sequences of the antibody heavy chains include at least 1 million amino acid sequences, at least 10 million amino acid sequences, at least 25 million amino acid sequences, at least 50 million amino acid sequences, at least 75 million amino acid sequences, at least 100 million amino acid sequences, at least 150 amino acid sequences, at least 200 million amino acid sequences, at least 250 million amino acid sequences, or at least 270 million amino acid sequences.

In some embodiments, training the at least one language model comprises training a light chain language model using light chain training data, the light chain training data comprising amino acid sequences of antibody light chains.

In some embodiments, the amino acid sequences of the antibody light chains include at least 1 million amino acid sequences, at least 10 million amino acid sequences, at least 20 million amino acid sequences, at least 30 million amino acid sequences, at least 40 million amino acid sequences, at least 50 million amino acid sequences, at least 60 million amino acid sequences or at least 70 million amino acid sequences.

In some embodiments, training the at least one language model comprises training a paired heavy-light chain language model using paired heavy-light chain training data, the paired heavy-light chain training data comprising pairs of antibody heavy chain amino acid sequences and antibody light chain amino acid sequences.

In some embodiments, the pairs of the antibody heavy chain amino acid sequences and the antibody light chain amino acid sequences include at least 1 million pairs, at least 10 million pairs, at least 15 million pairs, at least 20 million pairs, at least 25 million pairs, or at least 30 million pairs.

In some embodiments, training the paired heavy-light chain model comprises providing, as input to the paired heavy-light chain model, a concatenation of an amino acid sequence of an antibody heavy chain and an amino acid sequence of an antibody light chain.

In some embodiments, the training data comprises a plurality of amino acid sequences, and wherein obtaining the training data using the candidate amino acid sequence comprises introducing mutations into the candidate amino acid sequence of the candidate antibody to obtain the plurality of amino acid sequences.

In some embodiments, the plurality of amino acid sequences includes amino acid sequences of antibody light chains and amino acid sequences of antibody heavy chains.

In some embodiments, training the machine learning model to predict the binding affinities between the antibodies and the target comprises: training a first machine learning model using the amino acid sequences of the antibody heavy chains to predict a first set of binding affinities between the antibodies and the target; and training a second machine learning model using the amino acid sequences of the antibody light chains to predict a second set of binding affinities between the antibodies and the target.

In some embodiments, training the machine learning model to predict the binding affinities between the antibodies and the target comprises fine-tuning the at least one trained language model using the training data to predict the binding affinities between the antibodies and the target.

In some embodiments, fine-tuning the at least one trained language model comprises: processing the training data using the at least one trained language model to obtain an intermediate output; and training at least one regression model using the intermediate output to predict the binding affinities between the antibodies and the target.

In some embodiments, fine-tuning the at least one trained language model comprises: processing the training data using the at least one trained language model to obtain an intermediate output; and training a probabilistic model using the intermediate output to predict the binding affinities between the antibodies and the target.

In some embodiments, processing the training data using the at least one trained language model to obtain the intermediate output comprises processing an amino acid sequence included in the training data using the at least one trained language model to obtain vector representations of amino acids in the amino acid sequence. Some embodiments further comprise: concatenating the vector representations of the amino acids; performing principal component analysis to reduce dimensions of the vector representations to obtain reduced vector representations; and training the probabilistic model using the reduced vector representations.

Some aspects provide for a method for designing proteins for binding to a target, the method comprising: obtaining an amino acid sequence of a candidate protein wherein the candidate protein binds to the target with a candidate binding affinity; determining, for proteins in a set of proteins, probabilities that binding affinities between the proteins and the target are greater than the candidate binding affinity, the proteins in the set of proteins having different amino acid sequences, the proteins including a first protein having a first amino acid sequence, and the probabilities including a first probability that a first binding affinity between the first protein and the target is greater than the candidate binding affinity, wherein determining the first probability comprises: processing the first amino acid sequence of the first protein using a trained machine learning model to obtain a first output indicative of the first binding affinity between the first protein and the target; and determining the first probability using the first output indicative of the first binding affinity between the first protein and the target; and identifying a subset of the set of proteins based on the determined probabilities that the binding affinities are greater than the candidate binding affinity.

### BRIEF DESCRIPTION OF DRAWINGS

Various aspects and embodiments of the disclosure provided herein are described below with reference to the following figures. The accompanying drawings are not intended to be drawing to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for designing proteins (e.g., antibodies, single-chain variable fragments (scFvs), etc.) for binding to a target, according to some embodiments of the technology described herein.

FIG. 1C is a diagram of an illustrative technique for training a machine learning model to predict binding affinities between proteins and a target, according to some embodiments of the technology described herein.

FIG. 2 is a block diagram of an example system 200 for predicting binding affinities and for designing proteins for binding to a target, according to some embodiments of the technology described herein.

FIG. 3 is a flowchart of an illustrative process 300 for designing proteins for binding to a target, according to some embodiments of the technology described herein.

FIG. 4 is a flowchart of an illustrative process 400 for training a machine learning model to predict binding affinities, according to some embodiments of the technology described herein.

FIG. 5A and FIG. 5B depict an example of a technique for designing antibodies for binding to a target, according to some embodiments of the technology described herein.

FIG. 6A and FIG. 6B show the empirical binding distribution of training data and designed single-chain variable fragments, according to some embodiments of the technology described herein.

FIG. 7A, FIG. 7B, and FIG. 7C show results of evaluating a machine learning model trained to predict binding affinities of amino acid sequences, according to some embodiments of the technology described herein.

FIG. 8A and FIG. 8B show distributions of diversity metrics determined for antibody heavy chain variants, according to some embodiments of the technology described herein.

FIG. 9A and FIG. 9B show distributions of diversity metrics determined for antibody light chain variants, according to some embodiments of the technology described herein.

FIG. 10A depicts summary statistics and an empirically measured affinity distribution of antibody heavy chain designs, according to some embodiments of the technology described herein.

FIG. 10B shows the percent of designed antibody heavy chain sequences that have a stronger empirical binding affinity than the candidate antibody, according to some embodiments of the technology described herein.

FIG. 10C compares diversity among multiple antibody heavy chain libraries, according to some embodiments of the technology described herein.

FIG. 10D depicts summary statistics and an empirically measured affinity distribution of antibody light chain designs, according to some embodiments of the technology described herein.

FIG. 10E shows the percent of designed antibody light chain sequences that have a stronger empirical binding affinity than the candidate antibody, according to some embodiments of the technology described herein.

FIG. 10F compares diversity among multiple antibody light chain libraries, according to some embodiments of the technology described herein.

FIG. 11 is a histogram comparing affinities empirically measured for antibody heavy chain designs with affinities empirically measured for antibody light chain designs, according to some embodiments of the technology describe herein.

FIG. 12A shows that antibodies identified according to embodiments of the technology described herein have greater median binding affinities than antibodies identified using conventional techniques.

FIG. 12B shows that, compared to antibodies identified using conventional techniques, a larger percentage of the antibodies identified according to embodiments of the technology described herein are stronger binders than a candidate antibody.

FIG. 12C shows the mutational variability of the antibodies identified according to some embodiments of the technology described herein.

FIG. 13A shows that antibodies identified according to embodiments of the technology described herein have greater median binding affinities than antibodies identified using conventional techniques.

FIG. 13B shows that, compared to antibodies identified using conventional techniques, a larger percentage of the antibodies identified according to embodiments of the technology described herein are stronger binders than a candidate antibody.

FIG. 13C shows the mutational variability of the antibodies identified according to some embodiments of the technology described herein.

FIG. 14A and FIG. 14B show that, compared to conventional techniques, amino acid sequences of the antibodies identified according to the techniques developed by the inventors are further away from the amino acid sequence of the candidate antibody.

FIG. 15A shows the performance of machine learning models trained to predict binding affinities of antibodies, according to embodiments of the technology described herein.

FIG. 15B and FIG. 15C show the mutational distance between an amino acid sequence of a candidate antibody and amino acid sequences of antibodies identified according to embodiments of the technology described herein.

FIG. 16 shows the estimated percent of sequences having a better binding performance than a threshold value (i.e., an estimated percent of success), according to some embodiments of the technology described herein.

FIG. 17A and FIG. 17B show, for antibody heavy chain variants designed according to embodiments of the technology described herein, an estimated percent of success matches well to an empirically measured percent of success.

FIGS. 18A and 18B show, for antibody light chain variant designed according to embodiments of the technology described herein, an estimated percent of success matches well to an empirically measured percent of success.

FIGS. 19A and 19B show that designing all complementary determining regions (CDRs) of an antibody heavy chain produces antibodies with higher estimated percents of success and diversity, according to some embodiments of the technology described herein.

FIG. 20 shows an example of masked language modeling with a bidirectional encoder representation from transformers (BERT) transformer, according to some embodiments of the technology described herein.

FIG. 21A and FIG. 21B show isoelectric points and hydrophilicities of heavy and light chain sequences, according to some embodiments of the technology described herein.

FIG. 22 is a schematic diagram of an illustrative computing device with which aspects described herein may be implemented.

### DETAILED DESCRIPTION

The inventors have developed techniques for designing proteins (e.g., antibodies, scFvs, etc.) having a predetermined characteristic. For example, in some embodiments, the techniques include designing proteins for binding to a target. In some embodiments, this includes: (a) obtaining an amino acid sequence of a candidate protein that binds to the target with a candidate binding affinity; (b) determining, for proteins in a set of proteins, probabilities that binding affinities between the proteins and the target are greater than or equal to a threshold (e.g., the candidate binding affinity); and (c) identifying a subset of the set of proteins based on the determined probabilities. In some embodiments, determining a probability that a binding affinity between the target and a protein is greater than or equal to the threshold includes: processing an amino acid sequence of the protein using a trained machine learning model to obtain an output indicative of the binding affinity; and using the output to determine the probability that the binding affinity is greater than or equal to the threshold.

Therapeutic proteins are an important and rapidly growing drug modality. Because the vast search space of protein sequences renders exhaustive evaluation of the entire protein space infeasible, screening relatively small numbers of proteins from synthetic generation, animal immunizations or human donors are used to identify candidate proteins. The screened library represents a small portion of the overall search space, and the resultant candidate proteins are often weak binders or suffer from developability issues.

Due to the combinatorial scaling of sequence space, stepwise, iterative approaches are conventionally used to optimize protein binding against target molecules. This involves designing and producing prospective proteins, and then experimentally measuring characteristics of the proteins to determine whether the measured characteristics satisfy design criteria. Such conventional approaches are time consuming, and effort is wasted interrogating nonfunctional proteins. For example, a protein having an improved binding characteristics may have other, unfavorable properties (e.g., hydrophobicity). Such proteins may require alterations to improve the unfavorable properties, but such alterations can negatively influence the previous optimized binding, resulting in additional measurement and engineering cycles.

Some conventional techniques utilize machine learning to assist in designing proteins. For example, such techniques include using a general purpose pre-trained generative language model for designing protein libraries that display good physical properties but are not target-specific and are highly similar to other conventional libraries that are already based on natural protein repertoires. Furthermore, none of the conventional machine-learning approaches allow the evaluation of designed protein libraries prior to experimentation, resulting in effort wasted on experimentally evaluating failed designs.

Accordingly, the inventors have developed techniques that address the above-described challenges associated with the conventional techniques for optimizing protein (e.g., antibody, scFv, etc.) binding against a target. In some embodiments, the techniques include: (a) obtaining an amino acid sequence of a candidate protein that binds to the target with a candidate binding affinity; (b) determining, for proteins in a set of proteins, probabilities that binding affinities between the proteins and the target are greater than or equal to a threshold (e.g., the candidate binding affinity); and (c) identifying a subset of the set of proteins based on the determined probabilities. In some embodiments, determining the probability that a binding affinity between a protein and the target is greater than or equal to the threshold includes: processing an amino acid sequence of the protein using a trained machine learning model to obtain an output indicative of the binding affinity between the protein and the target; and determining the probability using the output of the trained machine learning model. Because the techniques, in some embodiments, rely on sequence data (e.g., amino acid sequences of the proteins) without requiring sequence alignment data or knowledge of the target structure, they are particularly useful for early-stage protein development for any suitable target (e.g., target antigen).

In some embodiments, the techniques developed by the inventors further include using optimization (e.g., Bayesian optimization) to design proteins (e.g., antibodies, scFvs, etc.) in the set of proteins for which the probabilities (e.g., probabilities that binding affinities of the proteins are greater than the candidate binding affinity) are determined. In some embodiments, the optimization is performed to maximize the posterior probability that the binding affinity is greater than or equal to the threshold. In some embodiments, performing the optimization includes using a sampling algorithm (e.g., hill climb, genetic algorithm, Gibbs sampling, etc.) to generate amino acid sequences of the proteins. The use of a sampling algorithm enables the generation of sequences with high diversity, resulting in proteins that are strong binders, which have diverse properties. Designing protein libraries that are diverse allows for the selection of multiple preclinical candidates, uncorrelated in their downstream failure mode, such that if one fails, the entire pipeline is not likely to fail for the same reason.

In some embodiments, the inventors have further developed techniques for evaluating the designed proteins in silico. In some embodiments, this includes determining a metric indicative of the estimated percent of proteins in the identified subset of proteins having a better binding performance than a threshold value (e.g., the binding affinity of the candidate protein). By evaluating the proteins in silico, downstream experimentation can be avoided for proteins that are likely to be weak binders, thereby increasing the efficiency of the protein design process and preserving resources for experimentally measuring proteins that are strong binders and/or have characteristic meeting design criteria.

In some embodiments, the techniques developed by the inventors additionally, or alternatively include training a machine learning model to predict binding affinities between proteins and a target. In some embodiments, the techniques for training the machine learning model include: (a) training at least one language model to encode amino acid sequences; (b) obtaining training data using a candidate amino acid sequence of a candidate protein; and (c) training the machine learning model to predict the binding affinities between the proteins and the target using the at least one trained language model and the obtained training data. In some embodiments, training the machine learning model using the training data and the trained language model includes fine-tuning the trained language model using the training data. Because the fine-tuning leverages the learned knowledge from the pre-trained language model, the fine-tuning does not require a significant amount of training data to accurately and reliably predict binding affinity against the target. Furthermore, because the training data is obtained using the candidate protein, and the candidate protein binds to the target, the training data is target specific. Accordingly, training the machine learning model using the target-specific training data enables binding affinity predictions that are target specific, and therefore more accurate and precise than conventional techniques which are not target specific.

Following below are descriptions of various concepts related to, and embodiments of, techniques for designing proteins for binding to a target. It should be appreciated that various aspects described herein may be implemented in any of numerous ways, as the techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

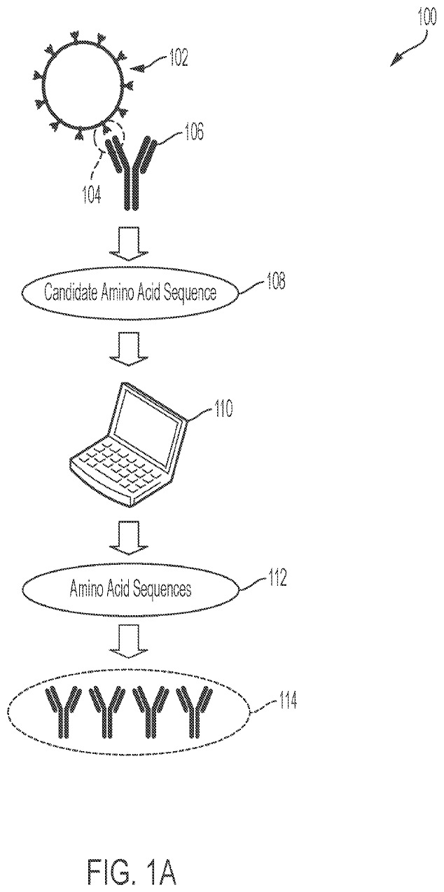

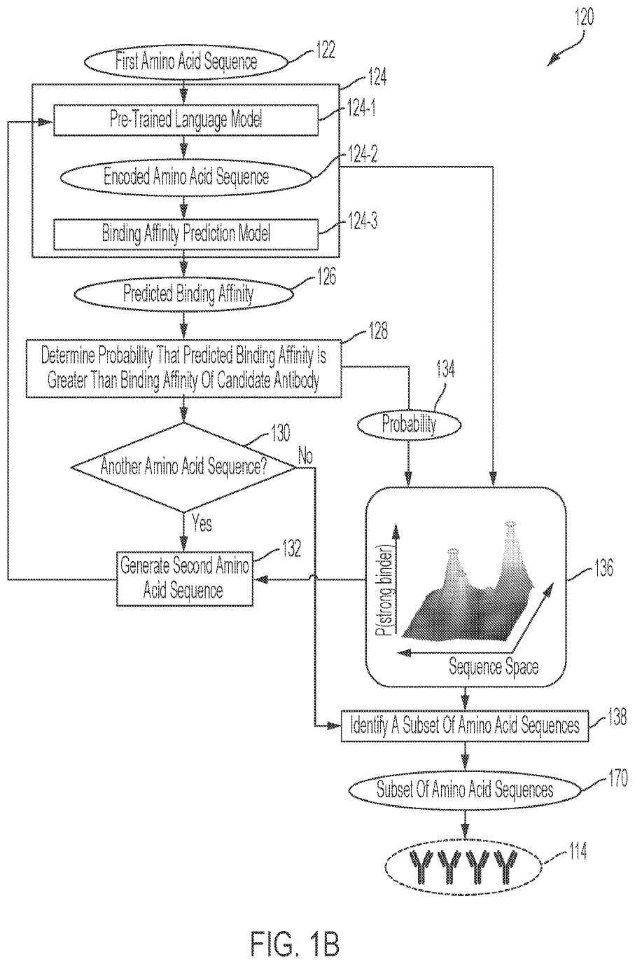

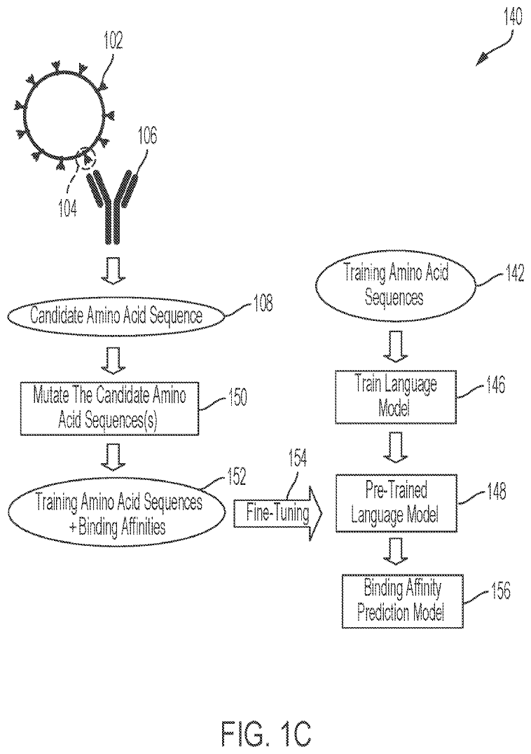

FIG. 1A is a diagram of an illustrative technique 100 for designing proteins (e.g., antibodies, scFvs, etc.) for binding to a target, according to some embodiments of the technology described herein. Technique 100 includes obtaining a candidate amino acid sequence 108 for a candidate protein 106 that binds to target 104 with a candidate binding affinity and processing the candidate protein sequence 108 using computing device 110 to obtain amino acid sequences 112 for proteins predicted to bind to the target with a binding affinity greater than a threshold (e.g., greater than the candidate binding affinity). In some embodiments, technique 100 further includes using at least some of the amino acid sequences 112 to produce proteins 114 for further evaluation, for therapeutic administration, or for any other suitable purpose, as aspects of the technology described herein are not limited in this respect.

In some embodiments, aspects of the illustrated technique 100 may be implemented in a clinical, laboratory, or protein manufacturing setting. For example, aspects of the illustrated technique 100 may be implemented on computing device 110 that is located within a clinical, laboratory, or protein manufacturing setting. In some embodiments, aspects of the illustrated technique 100 may be implemented in a setting that is located externally from a clinical, laboratory, or protein manufacturing setting. In this case, the computing device 110 may indirectly obtain the candidate amino acid sequence 108 from a device (e.g., a computing device) located within or externally to a clinical, laboratory, or protein manufacturing setting. For example, the candidate amino acid sequence 108 via at least one communication network, such as the Internet or any other suitable communication network(s), as aspects of the technology described herein are not limited in this respect.

As shown in FIG. 1A, the candidate amino acid sequence 108 is obtained for a candidate protein 106. The candidate amino acid sequence 108 may include or more amino acid sequences for one or more candidate proteins (e.g., candidate protein 106). In some embodiments, the candidate protein 106 is a protein that has been identified as having binding characteristics that meet one or more binding criteria. For example, the candidate protein 106 may include a protein that binds to a target 104 with a binding affinity that is greater than or equal to a binding affinity threshold. The binding affinity threshold may include any suitable threshold, as aspects of the technology are not limited in this respect.

In some embodiments, the candidate protein 106 is identified using any suitable techniques, as aspects of the technology described herein are not limited in this respect. As a non-limiting example, a phage display campaign with a phage library containing fragment antigen-binding (Fabs) regions of antibodies may be used to identify one or more candidate amino acid sequences that bind to the target.

The target 104 may include any suitable target, as aspects of the technology described herein are not limited in this respect. For example, the target 104 may be a portion of a foreign molecule 102 such as a foreign protein or antigen, that is capable of stimulating an immune response. In the example described with respect to the “Examples” section, the target is a conserved sequence found in the HR2 region of coronavirus spike proteins.

In some embodiments, a computing device 110 is used to process the candidate amino acid sequence 108 to obtain the amino acid sequences 112. The computing device 110 may be operated by a user such as a researcher, a manufacturer, a doctor, and/or any other suitable entity. For example, the user may provide the candidate amino acid sequence 108 as input to the computing device 110 (e.g., by uploading a file), provide user input specifying processing of other methods to be performed using the candidate amino acid sequence 108, and/or provide input specifying a binding affinity between the candidate protein 106 and the target 104.

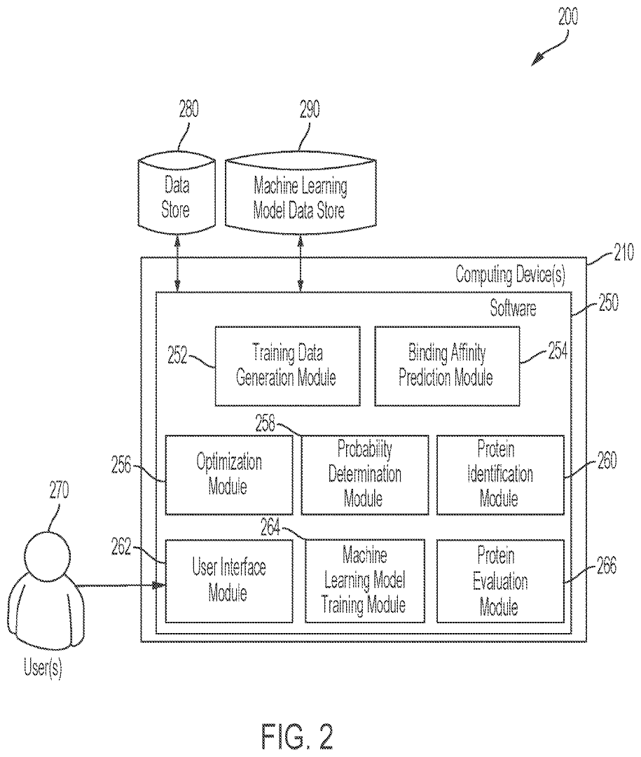

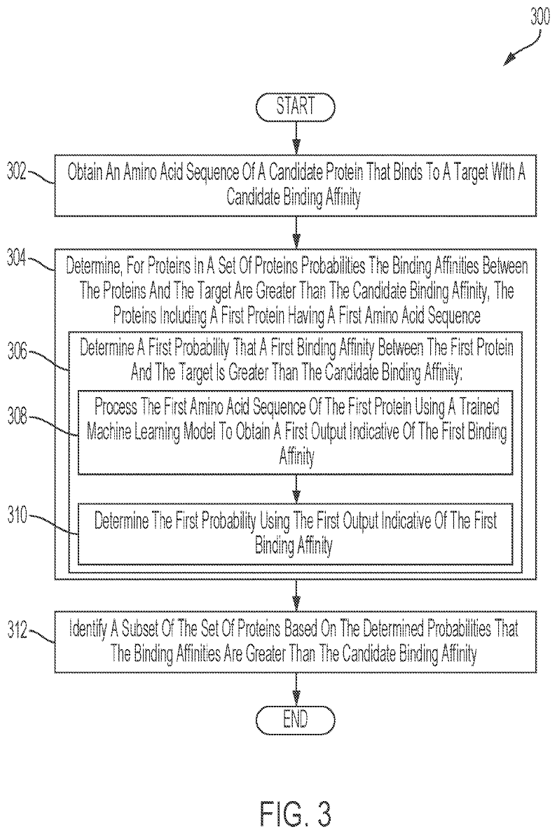

In some embodiments, software on the computing device 110 may be used to identify amino acid sequences 112 for proteins that are predicted to bind to the target 104 with a binding affinity greater than or equal to a threshold binding affinity (e.g., greater than or equal to the binding affinity of the candidate protein 106). An example of the computing device 110 and such software is described herein including at least with respect to FIG. 2 (e.g., computing device(s) 210 and software 250). In some embodiments, software on the computing device 110 may be configured to process the candidate amino acid sequence to identify amino acid sequences 112. In some embodiments, this includes: (a) generating amino acid sequences for a set of proteins (e.g., using optimization/sampling), (b) determining probabilities that binding affinities between proteins in the set of proteins and the target 104 are greater than or equal to a threshold binding affinity, and (c) identifying a subset of the set of proteins based on the determined probabilities. In some embodiments, to determine the probabilities, the computing device 110 is configured to process the generated amino acid sequences using a trained machine learning model to predict the binding affinities between the proteins and the target and determine the probabilities using the binding affinity predictions. Example techniques for identifying amino acid sequences for proteins are described herein including at least with respect to FIG. 1B and FIG. 3.

In some embodiments, software on the computing device 110 may additionally, or alternatively be used to evaluate amino acid sequences (e.g., amino acid sequences 112) generated according to embodiments of the technology described herein. In some embodiments, this includes determining a metric indicative of the estimated percent of proteins in the identified subset of proteins having a better binding performance than a threshold value (e.g., the binding affinity of the candidate protein).

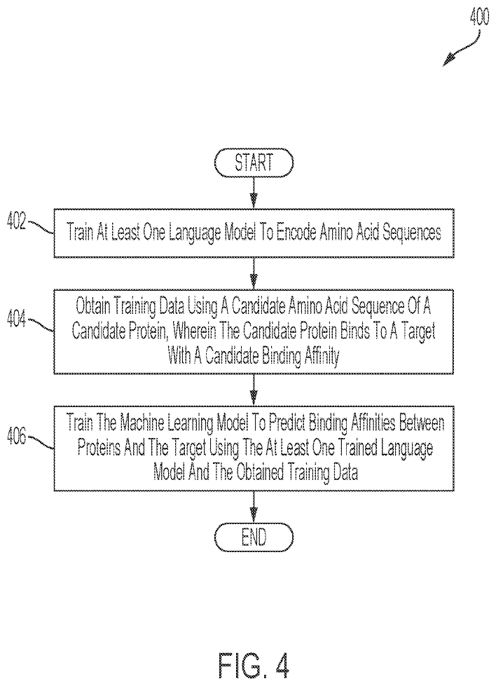

In some embodiments, software on the computing device 110 may additionally, or alternatively be used to train a machine learning model to predict a binding affinity between a protein and a target. In some embodiments, this includes training a language model to encode amino acid sequences and fine-tuning the language model to predict binding affinities for input amino acid sequences. Additionally, or alternatively, this may include obtaining a pre-trained language model, and fine-tuning the pre-trained language model to predict binding affinities for input amino acid sequences. Example techniques for training a machine learning model to predict binding affinities for input amino acid sequences are described herein including at least with respect to FIG. 1C and FIG. 4.

In some embodiments, the computing device 110 is configured to generate an output indicating one or more amino acid sequences 112. For example, in some embodiments, the output may indicate one or more amino acid sequences 112 identified as having a binding affinity that is greater than or equal to a threshold binding affinity. In some embodiments, the output may be indicative of a binding affinity predicted for the one or more amino acids. For example, an output indicative of a binding affinity may include an average and standard deviation of a binding affinity. Additionally, or alternatively, an output indicative of a binding affinity may include a measure of a number of binding interactions, which may be used to approximate binding affinity. In some embodiments, the output may further indicate a probability that the binding affinity predicted for the one or more amino acid sequences is greater than or equal to the threshold binding affinity. Additionally, or alternatively, the output may indicate one or more other properties associated with the amino acid sequence (e.g., hydrophilicity/hydrophobicity, etc.).

In some embodiments, the output of the computing device 110 is stored (e.g., in memory), displayed via a user interface, transmitted to one or more other devices, used to generate a report, or otherwise processed using any other suitable techniques, as aspects of the technology described herein are not limited in this respect. For example, the output of the computing device 110 may be displayed using a graphical user interface (GUI) of a computing device (e.g., computing device 110).

In some embodiments, the output is (optionally) is used to produce one or more proteins 114 having the amino acid sequences 112. In some embodiments, the proteins 114 may be evaluated using one or more experimental techniques, used in one or more protein therapies, or used in any other suitable manner, as aspects of the technology described herein are not limited in this respect.

In some embodiments, the computing device 110 includes one or multiple computing devices. In some embodiments, when the computing device 110 includes multiple computing devices, each of the computing devices may include software used to implement process 300 shown in FIG. 3 and/or process 400 shown in FIG. 4. In some embodiments, when the computing device 110 includes multiple computing devices, the computing devices may be used to perform different processes or different aspects of a process. For example, one computing device may include software used to implement process 300 shown in FIG. 3, while a different computing device may include software used to implement process 400 shown in FIG. 4.

In some embodiments, when the computing device 110 includes multiple computing devices, the multiple computing devices may be configured to communicate via at least one communication network such as the Internet or any other suitable communication network(s), as aspects of the technology described herein are not limited in this respect. For example, one computing device may be configured to train a machine learning model, and then provide the trained machine learning model to one or more other computing devices via the communication network.

FIG. 1B is a diagram of an illustrative technique for designing proteins for binding to a target, according to some embodiments of the technology described herein. As shown in FIG. 1B, the technique 120 includes processing a first amino acid sequence 122 using a trained machine learning model 124 to predict a binding affinity 126 for the first amino acid sequence, which is used to determine, at act 128, a probability 134 that the predicted binding affinity is greater than or equal to a threshold (e.g., the binding affinity between a candidate protein (e.g., candidate protein 106) and a target (e.g., target 104)). The probability 134 is used to construct fitness landscape 136. In some embodiments, at act 130, technique 120 may include determining whether to generate another amino acid sequence. Upon determining that another amino acid sequence is to be generated, technique 120 may include generating a second amino acid sequence (e.g., using a sampling algorithm), and processing the second amino acid sequence to determine a probability that the binding affinity between the second amino acid sequence and the target is greater than or equal to the threshold. Upon determining that another amino acid sequence is not to be generated, technique 120 may include using the optimized fitness landscape 136, at act 138, to identify a subset of amino acid sequences 170. The subset of amino acid sequences may be used to produce the one or more proteins 114.

The first amino acid sequence 122 may include any suitable amino acid sequence identified using any suitable techniques. For example, in some embodiments, the first amino acid sequence 122 is derived from an amino acid sequence used to train the machine learning model 124 (e.g., a seed sequence). For example, the first amino acid sequence 122 may be derived from an amino acid sequence in the training data that has binding characteristics that meet one or more binding criteria. In some embodiments, deriving the first amino acid sequence 122 from a seed sequence includes mutating the seed sequence or a sequence for which a probability has previously been determined. In some embodiments, a sequence may be mutated or otherwise generated according to a sampling algorithm. Example sampling techniques are described with respect to act 132.

In some embodiments, the first amino acid sequence 122 is processed using trained machine learning model 124 to predict an output indicative of the binding affinity 126 between a protein having the first amino acid sequence 122 and a target (e.g., target 104 in FIG. 1A). In some embodiments, the trained machine learning model includes a pre-trained language model 124-1 trained to encode the first amino acid sequence to obtain the encoded amino acid sequence 124-2, which is used as input to the binding affinity prediction model 124-3. In some embodiments, the binding affinity prediction model 124-3 is trained to predict a binding affinity 126 for the first amino acid sequence 122. The prediction, in some embodiments, may include an average and standard deviation of the predicted binding affinity. Examples of the pre-trained language model 124-1 and the binding affinity prediction model 124-3 are described herein including at least with respect to FIG. 3. Example techniques for training a machine learning model to predict binding affinity 126 are described herein including at least with respect to FIG. 1C and FIG. 4.

In some embodiments, a fitness function is extrapolated from the machine learning model 124 and used to construct the fitness landscape 136. In some embodiments, the fitness function is defined to be a mapping from an amino acid sequence to a posterior probability,

$$f ( x ) = p ( aff ( x ) < σ \vert x ) , \qquad ( Equation 1 )$$

that the estimated binding affinity aff(x) of the sequence x is greater than the threshold σ. For example, the threshold σ may be the measured binding affinity of a candidate protein and/or an average of measured binding affinities of multiple candidate proteins.

In some embodiments, optimization is performed to sample amino acid sequences with the highest extrapolated fitness value f(x) (i.e., the highest probability that the binding affinity is greater than the threshold σ). For example, the fitness value for the first amino acid sequence may include probability 134 determined for the first amino acid sequence. The determined probability may be used, as part of performing the optimization, to construct fitness landscape 136 based on the fitness function.

In some embodiments, technique 120 includes determining, at act 130, whether another amino acid sequence is to be generated. For example, technique 120 may include determining that another sequence is to be generated if the fitness function has not yet been optimized.

If it is determined, at act 130, that another amino acid sequence is to be generated, then technique 120 proceeds to act 132 for generating another amino acid sequence. In some embodiments, generating another amino acid sequence includes sampling around a seed sequence or around the first amino acid sequence 122 to generate a second amino acid sequence. Any suitable sampling techniques may be used, as aspects of the technology described herein are not limited in this respect. Nonlimiting examples of sampling techniques include the hill climb (HC) algorithm, the Gibbs sampling algorithm, and the genetic algorithm (GA). Sampling techniques are further described herein including at least with respect to FIG. 3.

In some embodiments, one or more acts of technique 120 may be repeated to process the second amino acid sequence generated at act 132. For example, the second amino acid may be processed using the trained machine learning model 124 to predict a binding affinity 126 (e.g., an average and standard deviation) for the second amino acid sequence. In some embodiments, the predicted binding affinity 126 is used to determine, at act 128, the probability that the predicted binding affinity 126 is greater than a threshold (e.g., the binding affinity of a candidate protein). The determined probability 134 may be used to construct the fitness landscape 136.

If, at act 130, it is determined that another amino acid sequence is not to be generated, then technique 120 proceeds to act 138 for identifying a subset of amino acid sequences 170. In some embodiments, the subset is identified using fitness landscape 136. As described above, in some embodiments, the fitness landscape maps the amino acid sequences to posterior probabilities that their respective binding affinities are greater than a threshold (e.g., the binding affinity of a candidate protein). In some embodiments, identifying the subset of amino acid sequences using the fitness landscape includes identifying amino acid sequences corresponding to the highest probabilities. For example, this may include rank-ordering the generated amino acid sequences (e.g., the first amino acid sequence, a second amino acid sequence, etc.) based on their probabilities (i.e., fitness scores) and selecting a number of the amino acids that were ranked the highest.

As described herein, including at least with respect to FIG. 1A, technique 120 may include producing one or more proteins 114 in the identified subset of amino acid sequences 170.

FIG. 1C is a diagram of an illustrative technique for training a machine learning model to predict binding affinities between proteins and a target, according to some embodiments of the technology described herein. Technique 140 includes, (a) training a language model at act 146, using training amino acid sequences 142, to obtain pre-trained language model 148, and (b) fine-tuning the pre-trained language model 148 at act 154 using training amino acid sequences and binding affinities 152 derived from the candidate amino acid sequence 108. Additionally, or alternatively, though not shown, in some embodiments, pre-trained language model 148 may have been previously trained, and technique 140 may include simply obtaining the pre-trained language model.

The amino acid sequences 142 used to train the language model at act 146 may include any suitable amino acid sequences. For example, the training amino acid sequences 142 may include protein sequences, antibody heavy chain sequences, antibody light chain sequences, and/or paired heavy-light chain sequences. The amino acid sequences 142 may be obtained from any suitable source, as aspects of the technology described herein are not limited in this respect. For example, the amino acid sequences 142 may be obtained from the Pfam database and/or the Observed Antibody Space database. The Pfam database is described by El-Gebali, S., et al. (“The Pfam protein family database in 2019.” Nucleic Acids Res. 47, D427-D432 (2019).), which is incorporated by reference herein in its entirety. OAS is described by Kovaltsuk, A. et al. (“Observed Antibody Space: A Resource for Data Mining Next-Generation Sequencing of Antibody Repertoires. J. Immunol 201, 2502-2509 (2018).), which is incorporated by reference herein in its entirety.

In some embodiments, training the language model at act 146 includes training a masked language model to encode protein and/or antibody amino acid sequences (e.g., the protein and/or amino acid sequences included in training data 142). In some embodiments, the language model estimates the probability of an amino acid sequence p(x) by considering the probability distribution over each amino acid at each position conditioned on all other amino acids in the sequence (e.g., Equation 2), that is,

$$p ( x ) = {\prod }_{i - 1}^{i = L} p ( {x}_{i} \vert {x}_{1} … {x}_{i - 1} , {x}_{i + 1} … {x}_{L} ) \qquad ( Equation 2 )$$

where xᵢ represents the i^{th} amino acid in the sequence of length L. In some embodiments, the language model is trained to predict randomly masked amino acids in a single sequence or a sequence pair (e.g., a heavy-light chain pair).

In some embodiments, multiple language models are trained at act 146. For example, different language models may be trained using (a) the protein sequence data, (b) the antibody heavy chain sequence data, (c) the antibody light chain sequence data, and (d) the antibody heavy-light chain sequence data. For training the paired heavy-light chain language model, in some embodiments, the input may include a concatenation of the heavy and light chain sequences separated by a token indicative of the order of the heavy and light chain in the concatenation.

In some embodiments, the training amino acid sequences and binding affinities 152 are derived from candidate amino acid sequence 108. For example, as described above, the candidate amino acid sequence 108 may include multiple candidate amino acid sequences, for which binding affinities have been experimentally determined. The training amino acid sequences may include the candidate amino acid sequences and their corresponding binding affinities, as well as one or more amino acid sequences derived from the candidate amino acid sequences. For example, one or more amino acid sequences may be derived from the candidate amino acid sequences by performing one or more mutations on the candidate amino acid sequences at act 150. In some embodiments, the binding affinities for the mutated amino acid sequences may be experimentally measured and included in the training data 152.

At act 154 of technique 140, the pre-trained language model 148 is fine-tuned using training data 152 to obtain the trained binding affinity prediction model 156. Any suitable machine learning approach for predicting binding affinity may be used, as aspects of the technology described herein are not limited in this respect. For example, models trained using an ensemble method and/or a Gaussian Process method may be used to predict binding affinity. Both example approaches provided estimates of prediction uncertainties.

In some embodiments, the ensemble model includes multiple different trained regression models fine-tuned from the pre-trained language model(s) 148. In some embodiments, fine-tuning the pre-trained language model(s) according to the ensemble method includes adding a linear regression head to the pre-trained language model and continuing to train it on the training amino acid sequences and binding affinities 152. In some embodiments, the ensemble model outputs the mean and standard deviation of the outputs of the multiple regression models.

In some embodiments, fine-tuning the pre-trained language model(s) according to the Gaussian Process method includes (a) representing the sequences by first concatenating the learned vector representations of each amino acid from the pre-trained language model 148, and then performing principal component analysis (PCA) to reduce the vector dimension. The Gaussian Process model is trained on the reduced vector representations. In some embodiments, the statistical model outputs a mean and standard deviation of the binding affinity prediction.

In some embodiments, when the pre-trained language model 148 includes multiple pre-trained language models, each of the multiple pre-trained language models may be fine-tuned to obtain the trained binding affinity prediction model 156.

FIG. 2 is a block diagram of an example system 200 for predicting binding affinities and for designing proteins for binding to a target, according to some embodiments of the technology described herein. System 200 includes computing device(s) 210 configured to have software 250 execute thereon to perform various functions in connection with computationally designing proteins for binding to a target. In some embodiments, software 250 includes a plurality of modules. A module may include processor-executable instructions that, when executed by at least one computer hardware processor, cause the at least one computer hardware processor to perform the function(s) of the module. Such modules are sometimes referred to herein as “software modules,” each of which includes processor executable instructions configured to perform one or more processes, such as process 300 described herein including at least with respect to FIG. 3 and process 400 described herein including at least with respect to FIG. 4.

The computing device(s) 210 may be operated by user(s) 270. In some embodiments, the user(s) 270 may provide, as input to the computing device(s) 210 (e.g., by uploading one or more files, by interacting with a user interface of the computing device(s) 210, etc.) data about proteins (e.g., antibodies), a target (e.g., an antigen), or any other suitable data as aspects of the technology described herein are not limited in this respect. Data about the proteins may include, for example, amino acid sequences of the proteins, binding affinities between the proteins and a target, or any other suitable properties, as aspects of the technology described herein are not limited in this respect. Additionally, or alternatively, the user(s) 270 may provide input specifying processing or other methods to be performed on the data (e.g., processing the data using a machine learning model, training a machine learning model, generating training data, evaluating performance of a machine learning model, etc.). Additionally, or alternatively, the user(s) 270 may access results of processing the data. For example, the user(s) 270 access results indicative of one or more identified amino acid sequences and/or binding affinities predicted for one or more proteins.

As shown in FIG. 2, software 250 includes multiple software modules for predicting binding affinities and designing proteins (e.g., antibodies, scFvs, etc.) for binding to a target. Such software modules include a binding affinity prediction module 254, an optimization module 256, a probability determination module 258, a protein identification module 260, and a protein evaluation module 266.

In some embodiments, the optimization module 256 is configured to extrapolate, from a trained machine learning model, a fitness function over which to optimize. For example, the optimization module 256 may extrapolate the fitness function from a trained machine learning model stored in the machine learning model data store 290, a trained machine learning model obtained from the machine learning model training module 264, and/or a trained machine learning model obtained from user(s) 270. In some embodiments, as described herein including at least with respect to FIG. 1B and FIG. 3, the fitness function may be defined to be a mapping from an amino acid sequence to a posterior probability that the estimated binding affinity of the sequence is greater than a threshold.

In some embodiments, the optimization module 256 is additionally, or alternatively, configured to generate protein sequences by performing one or more sampling algorithms. Nonlimiting examples of sampling algorithms include a hill climb algorithm, a genetic algorithm, a Gibbs sampling algorithm, or any other suitable sampling algorithm, as aspects of the technology described herein are not limited in this respect. Examples of generating sequences using sampling techniques are described herein including at least with respect to process 300 shown in FIG. 3.

In some embodiments, the optimization module 256 is additionally, or alternatively, configured to construct a fitness landscape using solutions to the fitness function determined for the sequences generated by the optimization module 256. The fitness landscape, for example, may map sampled amino acid sequences to the posterior probability that the predicted binding affinity is stronger than a threshold binding affinity (e.g., the binding affinity of a candidate protein). In some embodiments, the optimization module 256 obtains said probabilities from the probability determination module 258, the data store 280, and/or the user(s) 270 (e.g., by uploading a file).

In some embodiments, the binding affinity prediction module 254 is configured to obtain an amino acid sequence from data store 280, user(s) 270 (e.g., by uploading a file), and/or from optimization module 256. In some embodiments, the binding affinity prediction module 254 obtains a trained machine learning model from machine learning model data store 290, from machine learning model training module 264, and/or from user(s) 270 (e.g., by uploading a file).

In some embodiments, the binding affinity prediction model is configured to process an amino acid sequence of a protein using a trained machine learning model to obtain an output indicative of a binding affinity between the protein and a target. In some embodiments, the output of the trained machine learning model includes an average and standard deviation of a predicted binding affinity for the amino acid sequence. Additionally, or alternatively, the output indicative of the binding affinity may include a measure of a number of binding interactions, which may be used to approximate binding affinity. Example machine learning models trained to predict binding affinities are described herein including at least with respect to FIGS. 1A-B, 3, and 4. Example techniques for training a machine learning model to predict binding affinities are described herein including at least with respect to FIG. 1C and FIG. 4.

In some embodiments, the probability determination module 258 is configured to obtain a binding affinity prediction for an amino acid sequence from the binding affinity prediction module 254, data store 280, and/or user(s) 270 (e.g., by uploading a file).

In some embodiments, the probability determination module 258 is configured to process a binding affinity prediction for a protein to determine a probability that the binding affinity is greater than or equal to a threshold. The probability may be determined using a mean and standard deviation of the predicted binding affinity for the protein. The threshold may include any suitable threshold such as, for example, the binding affinity measured for a candidate protein, or an average binding affinity determined for multiple candidate proteins.

In some embodiments, the protein identification module 260 is configured to obtain a fitness landscape from the optimization module 256, data store 280, and/or user(s) 270.

In some embodiments, the protein identification module 260 is configured to identify a subset of proteins or which binding affinities were predicted and/or probabilities were determined. For example, in some embodiments, the protein identification module 260 may use a fitness landscape, which maps amino acid sequences to posterior probabilities, to identify a subset of the set of proteins for which amino acid sequences were generated (e.g., by optimization module 256) and for which binding affinities/probabilities were predicted (e.g., by binding affinity prediction module 254 and probability determination module 158). In some embodiments, the protein identification module 260 is configured to identify the subset of proteins by rank-ordering the proteins based on the probabilities determined for their amino acid sequences and identifying the top-ranked proteins for inclusion in the subset. For example, the protein identification module 260 may identify the top N ranked proteins, where Nis any suitable number of proteins.

In some embodiments, the protein evaluation module 266 is configured to obtain a subset of proteins from the protein identification module 260, the data store 280, and/or user(s) 270.

In some embodiments, the protein evaluation module 266 is configured to quantify the binding performance of the identified subset of proteins prior to experimental testing. For example, in some embodiments, the protein evaluation module 266 is configured to determine a metric indicative of the probability of success (i.e., the estimated percent of proteins having a better binding performance than the threshold value). This may include averaging the fitness scores (i.e., the probabilities that the binding affinities between the proteins and the target are greater than or equal to a threshold) determined for the proteins.

In some embodiments, software 250 further includes software modules for training one or more machine learning models. Such software modules include the training data generation module 252 and machine learning model training module 264.

In some embodiments, training data generation module 252 is configured to generate training data for training a binding affinity prediction module. In this respect, the training data generation module 252 may be configured to obtain amino acid sequences of one or more candidate proteins and generate additional training data using the obtained sequences. For example, in some embodiments, the training data generation module 252 may be configured to perform mutations within obtained amino acid sequences (e.g., the amino acid sequences obtained for the one or more candidate proteins) to generate additional training sequences.

In some embodiments, the training data generation module 252 may be configured to provide the generated amino acid sequences to data store 280, user interface module 262, and/or machine learning model training module 264. For example, the generated amino acid sequences may be provided as output to a user and/or automated system, such that the user and/or automated system may produce the proteins having the generated amino acid sequences and measure binding affinities of said proteins. The measured binding affinities may be provided to software 250 for training a machine learning model (e.g., a binding affinity prediction model).

In some embodiments, the machine learning model training module 264 obtains training data from training data generation module 252, data store 280, and/or user(s) 270 (e.g., by uploading a file).

In some embodiments, the machine learning model training module 264 is configured to train at least one language model to encode amino acid sequences. In this respect, the machine learning model training module 264 may obtain training data that includes amino acid sequences for proteins, antibody light chains, antibody heavy chains, and/or antibody heavy-light chain pairs. In some embodiments, the machine learning model training module 264 is configured to train the language model(s) to predict randomly masked amino acids of an amino acid sequence or sequence pair. The language model may include any suitable language model such as, for example, a BERT masked language model.

Additionally, or alternatively, in some embodiments, the machine learning model training module 264 is configured to train a machine learning model to predict a binding affinity for an input amino acid sequence or an amino acid sequence pair. In this respect, the machine learning model training module 264 may be configured to obtain training data that includes amino acid sequences and their corresponding measured binding affinities and use the obtained training data to train the machine learning model to predict binding affinities of input amino acid sequences.

The machine learning model training module 264 may be configured to train the affinity prediction model according to any suitable techniques, as aspects of the technology described herein are not limited in this respect. For example, the machine learning model training module 264 may be configured to train the affinity prediction model according to an ensemble approach or a Gaussian Process approach. Example machine learning approaches are described herein including at least with respect to FIG. 1C, FIG. 3, and FIG. 4.

In some embodiments, the machine learning model training module 264 may provide trained machine learning model(s) to machine learning model data store 290 for storage therein. The machine learning model data store 290 may be of any suitable type (e.g., database system, multi-file, flat file, etc.) and may store machine learning model data in any suitable way and in any suitable format, as aspects of the technology described herein are not limited in this respect. The machine learning model data store 290 may be part of or external to computing device(s) 210.

In some embodiments, software 250 further includes user interface module 262. User interface module 262 may be configured to generate a graphical user interface through which a user may provide input and view information generated by software 250. For example, in some embodiments, the user interface may be a webpage or web application accessible through an Internet browser. In some embodiments, the user interface module 262 may generate a graphical user interface (GUI) of an app executing on the user's mobile device. In some embodiments, the user interface module 262 may generate a number of selectable elements through which a user may interact. For example, the user interface module 262 may generate dropdown lists, checkboxes, text fields, or any other suitable element.

In some embodiments, the user interface module 262 is configured to generate a GUI including one or more results of processing data such as, for example, data about a candidate protein and/or a target. For example, the GUI may include an indication of one or more amino acid sequences for proteins predicted to have a stronger binding affinity than a candidate protein. Additionally, or alternatively, the GUI may include indications of binding affinities predicted for amino acid sequences of different proteins. It should be appreciated that the GUI may include any other suitable information, displayed in any suitable manner, as aspects of the technology described herein are not limited in this respect.

System 200 further includes data store 280. In some embodiments, the data store 280 stores any suitable data, as aspects of the technology are not limited in this respect. For example, in some embodiments, the data store 280 stores one or more amino acid sequences and/or corresponding measured binding affinities used for training machine learning model(s) to encode amino acid sequences and/or predict binding affinities for amino acid sequences. Additionally, or alternatively, in some embodiments, the data store 280 stores the outputs of one or more software modules included in software 250. For example, the data store 280 may store training data generated by training data generation module 252, binding affinities output by binding affinity prediction module 254, a fitness function, fitness landscape, or generated sequences output by the optimization module 256, probabilities output by probability determination module 258, subset(s) of proteins output by protein identification module 260, and/or results of evaluating proteins output by the protein evaluation module 266.

The data store 280 may be of any suitable type (e.g., database system, multi-file, flat file, etc.) and may store data in any suitable way in any suitable way in any suitable format, as aspects of the technology described herein are not limited in this respect. The data store 280 may be part of or external to the computing device(s) 210.

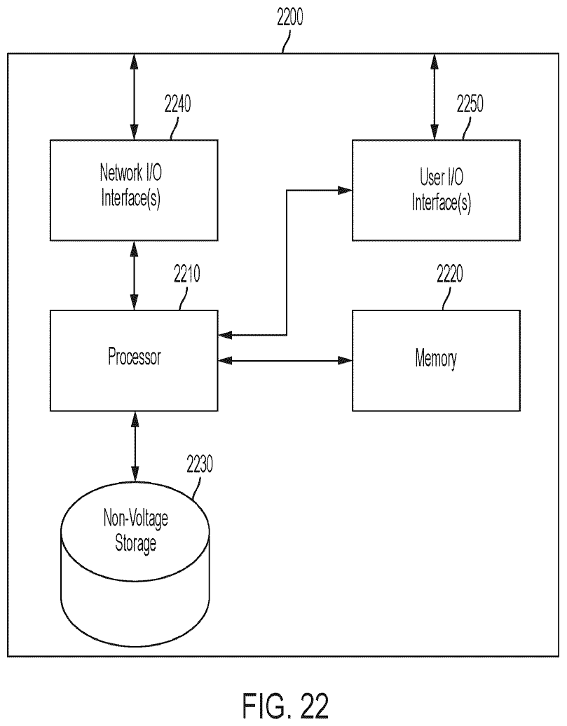

FIG. 3 is a flowchart of an illustrative process 300 for designing proteins (e.g., antibodies, scFvs, etc.) for binding to a target, according to some embodiments of the technology described herein. One or more acts of process 300 may be performed automatically by any suitable computing device(s). For example, the act(s) may be performed by a laptop computer, a desktop computer, one or more servers, in a cloud computing environment, computing device 2200 as described herein with respect to FIG. 22, and/or in any other suitable way.

At act 302, an amino acid sequence of a candidate protein that binds to a target with a candidate binding affinity is obtained. The amino acid sequence may include or more amino acid sequences for one or more candidate proteins.

In some embodiments, a candidate protein is a protein that has been identified as having binding characteristics that meet one or more binding criteria. For example, the candidate protein may include a protein that binds to a target with a binding affinity that is greater than or equal to a threshold. The binding affinity threshold may include any suitable threshold, as aspects of the technology are not limited in this respect.

In some embodiments, the candidate protein is identified using any suitable techniques, as aspects of the technology described herein are not limited in this respect. As a non-limiting example, a phage display campaign with a phage library containing fragment antigen-binding (Fabs) regions of antibodies may be used to identify one or more candidate amino acid sequences that bind to the target.

The target may include any suitable target, as aspects of the technology described herein are not limited in this respect. For example, the target may be a portion of a foreign molecule such as a foreign protein or antigen, that is capable of stimulating an immune response. In the example described with respect to the “Examples” section, the target is a conserved sequence found in the HR2 region of coronavirus spike proteins.

At act 304, probabilities are determined for proteins in a set of proteins. The probabilities are probabilities that binding affinities between the proteins and the target are greater than a threshold. For example, the threshold may be the candidate binding affinity measured for the candidate protein or, when the candidate protein includes multiple candidate proteins, an average of the candidate binding affinities measured for the candidate proteins.

In some embodiments, a probability may be determined for each protein in the set of proteins. For example, a first probability may be determined for a first protein in the set of proteins.

In some embodiments, the set of proteins includes proteins having amino acid sequences generated via optimization. Optimization, in some embodiments, may be performed to optimize a fitness function and construct a fitness landscape. In some embodiments, the fitness function is defined to be a mapping from an amino acid sequence to the posterior probability that the estimated binding affinity of a sequence is greater than the threshold (e.g., Equation 1). In some embodiments, the fitness function is extrapolated from a machine learning model trained to predict, for input amino acid sequences, the predicting binding affinities between proteins having the input amino acid sequences and the target.

In some embodiments, optimizing a fitness function includes generating an amino acid sequence in the set of amino acid sequences, determining a probability for the generated amino acid sequence, and either terminating the optimization or generating the next amino acid sequence in the set of amino acid sequences based on the determined probability. For example, a first probability may be determined for a first amino acid sequence in the set of amino acid sequences, at act 306, and the first probability may be used to determine whether to generate another amino acid sequence or proceed to act 312.

In some embodiments, the amino acid sequences in the set of amino acid sequences are generated using a sampling algorithm. The sampling algorithm may include any suitable sampling algorithm, as aspects of the techniques described herein are not limited in this respect. Nonlimiting examples of sampling algorithms include a hill climb algorithm, a genetic algorithm, and a Gibbs sampling algorithm. The hill climb algorithm is described by Russel, S. and Norvig, P. (“Artificial Intelligence: A Modern Approach,” 4^{th} U.S. ed., (2022)), which is incorporated by reference herein in its entirety. The genetic algorithm is described by Katoch, S., Chauhan, S., and Kumar, V. (“A review on genetic algorithm: past, present, and future,” Multimed. Tools Appl 80, 8091-8126 (2021)), which is incorporated by reference herein in its entirety. Gibbs sampling algorithm is described by Levine, R. et al. (“Implementing random scan Gibbs samplers,” Comput. Stat. 20, 177-196 (2005).), which is incorporated by reference herein in its entirety.

In some embodiments, the sampling algorithm(s) may be initialized using any suitable amino acid sequence(s) (e.g., seed sequence(s)). The seed sequences may include amino acid sequences used to train a machine learning model to predict binding affinities, such as the machine learning model described herein including at least with respect to act 308. For example, the seed sequences may be selected based on binding affinities experimentally measured for proteins. For example, amino acid sequences corresponding to proteins measured to have the strongest binding affinities may be used as seed sequences. Any suitable number of seed sequences may be used, as aspects of the technology are not limited in this respect. For example, at least 6 amino acid sequence, at least 8 amino acid sequences, at least 10 amino acid sequences, at least 12 amino acid sequences, or at least 14 amino acid sequences may be used as seed sequences.

The hill climb algorithm may be implemented in any suitable manner, as aspects of the technology are not limited in this respect. For example, the hill climb algorithm may be initialized by randomly mutating a seed sequence with a predetermined number of mutations (e.g., 1 mutation, 2 mutations, 3 mutations, 4 mutations, etc.). At each step, the hill climb algorithm may perform a local search around the current sequence and sample the next sequence that has the highest fitness value (e.g., the highest posterior probability that the binding affinity is greater than or equal to a threshold). The search may continue until it can no longer find an amino acid sequence that has a higher fitness value than the current sequence. The local search space may be defined to be any suitable number of mutants of the current sequence, as aspects of the technology are not limited in this respect. For example, the local search space may be defined to be 1000 mutants of the current sequence, including k=1 mutations and random k=2 mutations. The hill climb algorithm may be run any suitable number of times, as aspects of the technology are not limited in this respect. For example, the hill climb algorithm may be run 100 times with random restart around a random seed sequence.

The genetic algorithm may be implemented in any suitable manner, as aspects of the technology are not limited in this respect. For example, a population may be initialized with a random seed sequence selected from amino acid sequences of proteins measured to have the strongest binding affinities of multiple amino acid sequences. Parents may be chosen from the current population using any suitable model of evolution, such as, for example, the Wright-Fisher model of evolution where member of the current population become parents with a probability exponential to their fitness value. The Wright-Fisher model is described by Tran, T. et al. (“An introduction to the mathematical structure of the Wright-Fisher model of population genetics.” Theory Biosci. 132, 73-82 (2013).), which is incorporated by reference herein in its entirety. A single-point crossover may be performed on two parent sequences selected (e.g., randomly) from the parent population, and followed by randomly mutating individual child sequences with an expected mutation (e.g., k=1 mutation). In some embodiments, the algorithm is terminated when it no longer produces new sequences (i.e., it converges). The algorithm may be run any suitable number of times, as aspects of the technology described herein are not limited in this respect. For example, the genetic algorithm may be run 100 times, each initialized from a random seed sequence.

The Gibbs sampling algorithm may be implemented in any suitable manner, as aspects of the technology described herein are not limited in this respect. For example, the algorithm may be initialized from a seed sequence having the strongest binding affinity in the training data. At each step, a position i may randomly be selected, a mutant [symbol] may be sampled at the selected position with a conditional probability, and the sequence may be updated by replacing the i^{th} token with the sampled token [symbol] . The conditional probability may be defined in any suitable way, as aspects of the technology described herein are not limited in this respect. For example, the conditional probability may be defined to be exponential to the fitness values. The algorithm may be run any suitable number of times with any suitable number of iterations, as aspects of the technology described herein are not limited in this respect. For example, the algorithm may be run once with 30,000 iterations.

In some embodiments, determining the probabilities for the proteins includes, at act 306, determining a first probability that a first binding affinity between the first protein and the target is greater than the candidate binding affinity.

Determining the first probability includes, at act 308, processing a first amino acid sequence of the first protein using a trained machine learning model to obtain a first output indicative of the first binding affinity. The trained machine learning model may include any suitable machine learning model trained to predict, for an input amino acid sequence, the binding affinity between a protein having the amino acid sequence and the target, as aspects of the technology described herein are not limited in this respect. For example, the machine learning model may include a regression model including, for example, a linear regression model, a generalized linear model, a Gaussian Process model, a support vector machine model, a decision tree model, an ensemble model, or any other suitable machine learning model as aspects of the technology described herein are not limited in this respect. Example machine learning models and example techniques for training such models are described herein including at least with respect to FIG. 4.

At act 310, the output of the machine learning model is used to determine the probability that a binding affinity between the first protein and the target is greater than or equal to the threshold. For example, in some embodiments, the output of the machine learning model includes an average and standard deviation of the binding affinity estimated for the first protein, and the average and standard deviation may be used to determine the probability.

At act 312, a subset of the set of proteins is identified based on the probabilities determined at act 304. In some embodiments, this includes ranking the proteins in the set of proteins according to the probabilities determined for the proteins at act 304. For example, the proteins may be ranked from highest to lowest probabilities. In some embodiments, identifying the subset of the proteins includes identifying a number of proteins assigned ranks corresponding to the highest probabilities (i.e., top-ranked proteins). Any suitable number of proteins may be identified for inclusion in the subset, as aspects of the technology described herein are not limited in this respect. For example, the subset may include at least 1%, at least 2%, at least 4%, at least 6%, at least 8%, at least 10%, at least 15%, at least 20%, at least 25%, at least 30%, at least 40%, at least 50%, or any other suitable percentage of proteins included in the set of proteins, as aspects of the technology described herein are not limited in this respect. Additionally, or alternatively, the subset may include at most 5%, at most 10%, at most 25%, at most 20%, at most 25%, at most 30%, at most 40%, at most 50%, at most 60%, at most 70%, or at most any other suitable percentage of proteins included in the set of proteins, as aspects of the technology described herein are not limited in this respect.

It should be appreciated that process 300 may include one or more additional or alternative acts not shown in FIG. 3. For example, process 300 may include further processing the amino acid sequences for proteins identified for inclusion in the subset to determine one or more other characteristics of the proteins. Additionally, or alternatively, process 300 may include evaluating the identified subset of proteins to determine a metric indicative of the estimated percent of proteins having a better binding performance than the threshold value. Additionally, or alternatively, process 300 may include producing one or more of the proteins identified for inclusion in the subset.

FIG. 4 is a flowchart of an illustrative process 400 for training a machine learning model to predict binding affinities, according to some embodiments of the technology described herein. One or more acts of process 400 may be performed automatically by any suitable computing device(s). For example, the act(s) may be performed by a laptop computer, a desktop computer, one or more servers, in a cloud computing environment, computing device 2200 as described herein with respect to FIG. 22, and/or in any other suitable way.

At act 402, at least one language model is trained to encode amino acid sequences. In some embodiments, training the language model includes training a masked language model to encode protein and/or antibody amino acid sequences (e.g., the protein and/or amino acid sequences included in training data). In some embodiments, the language model estimates the probability of an amino acid sequence p(x) by considering the probability distribution over each amino acid at each position conditioned on all other amino acids in the sequence (e.g., Equation 2). In some embodiments, the language model is trained to predict randomly masked amino acids in a single sequence or a sequence pair (e.g., a heavy-light chain pair).

In some embodiments, the at least one language model is any suitable large language model, as aspects of the technology are not limited in this respect. For example, the large language model may include an autoencoding language model such as bidirectional encoder representations from transformers (BERT) model, or any other suitable autoencoding model, as aspects of the technology described herein are not limited in this respect. BERT is described by Devlin, J. et al. (“BERT: Pre-training of Deep Bidirectional Transformers for Language Understanding,” In Proceedings of the 2019 Conference of the North American Chapter of the Association for Computational Linguistics: Human Language Technologies, Vol 1, 4171-4186), which is incorporated by reference herein in its entirety.

As one non-limiting example, a BERT masked language model may be trained with 768 embedding size, 24 hidden layers, 1024 hidden size, 4096 immediate feedforward size, and 16 attention heads. Other architecture details may be fixed to their default values, as described by Devlin, J. et al., with Adam optimization. Adam optimization is described by Kingma, D. and Ba, J (“Adam: A Method for Stochastic Optimization,” arXiv preprint arXiv: 1412.6980 (2014)), which is incorporated herein by reference in its entirety.

The amino acid sequences used to train the language model may include any suitable amino acid sequences. For example, the amino acid sequences may include protein sequences, antibody heavy chain sequences, antibody light chain sequences, and/or paired heavy-light chain sequences. The amino acid sequences may be obtained from any suitable source, as aspects of the technology described herein are not limited in this respect. For example, the amino acid sequences may be obtained from the Pfam database and/or the Observed Antibody Space database.

In some embodiments, training the at least one language model at act 402 includes training multiple language models. For example, different language models may be trained using (a) the protein sequence data, (b) the antibody heavy chain sequence data, (c) the antibody light chain sequence data, and (d) the antibody heavy-light chain sequence data. For training the paired heavy-light chain language model, in some embodiments, the input may include a concatenation of the heavy and light chain sequences separated by a token indicative of the order of the heavy and light chain in the concatenation.

At act 404, training data is obtained using the candidate amino acid sequence of a candidate protein. For example, the candidate protein may include the one or more candidate proteins described herein including at least with respect to act 302 of process 300 shown in FIG. 3. In some embodiments, obtaining the training data includes obtaining the candidate amino acid sequence(s) themselves, together with the binding affinities measured for the candidate proteins having the candidate amino acid sequence(s). Additionally, or alternatively, obtaining the training data may include deriving one or more amino acid sequence(s) from the candidate protein sequence(s), and obtaining binding affinities for the proteins having the derived sequences. For example, one or more amino acid sequences may be derived from the candidate amino acid sequences by performing one or more mutations on the candidate amino acid sequences. In some embodiments, the binding affinities for the mutated amino acid sequences may be experimentally measured and included in the training data.

At act 406, a machine learning model is trained to predict binding affinities between proteins and the target using the at least one language model trained at act 402 and the training data obtained at act 404.

In some embodiments, as part of training the machine learning model, the obtained training data may be pre-processed according to any suitable techniques. For example, the training data may be split into different datasets (e.g., train, validation, and test sets) for training and evaluating the performance of the machine learning model. In some embodiments, the training data includes missing binding affinity values, and the missing values may be handled using any suitable approach. Two examples includes: dropping the assay with the missing value, or imputing it with the median value of all assays of the same candidate protein chain.

In some embodiments, training the machine learning model includes fine-tuning the pre-trained language model using the training data. Any suitable machine learning approach for training a model to predict binding affinity may be used, as aspects of the technology described herein are not limited in this respect. For example, models trained using an ensemble method and/or a Gaussian Process method may be used to predict binding affinity. Both example approaches provided estimates of prediction uncertainties.

In some embodiments, the ensemble model includes multiple different trained regression models fine-tuned from the pre-trained language model(s). For example, the multiple different trained regression models may include any suitable number of regression models and may depend on (a) the number of language models trained at act 402, (b) the loss functions used between the predicted and measured affinities, and/or (c) one or more pre-processing steps used. For example, a different regression model may be trained for each unique combination of pre-trained language, model, loss function, and pre-processing step. For example, when four language models are trained at act 402 (e.g., on protein sequences, light chain sequences, heavy chain sequences, and paired heavy-light chain sequences), two different loss functions are used (e.g., mean squared error and mean absolute error between the predicted and measured affinities), and two different data pre-processing steps (e.g., the two approaches for handling missing binding affinity values) are used, then 16 regression models may be included in the ensemble model.

In some embodiments, fine-tuning the pre-trained language model(s) according to the ensemble method includes adding a linear regression head to the pre-trained language model and continuing to train it on the amino acid sequences and binding affinities included in the training data. In some embodiments, the ensemble model outputs the mean and standard deviation of the outputs of the multiple regression models.

In some embodiments, fine-tuning the pre-trained language model(s) according to the Gaussian Process method includes (a) representing the sequences by first concatenating the learned vector representations of each amino acid from the pre-trained language model 148, and then performing principal component analysis (PCA) to reduce the vector dimension. For example, the vector dimension may be reduced to 1024. The Gaussian Process model is trained on the reduced vector representations. In some embodiments, the statistical model outputs a mean and standard deviation of the binding affinity prediction.

### APPLICATIONS

Embodiments of the technology described herein may be used to predict binding affinities between proteins and a target and to further design proteins having binding affinities that meet binding criteria. For example, as described above with respect to FIGS. 1A-4, embodiments of the technology described herein may be used to design proteins that bind to a target with a binding affinity that is stronger than the binding affinity between a candidate protein sequence and the target. However, it should be appreciated that the techniques developed by the inventors are not limited to predicting binding affinities and designing proteins having binding affinities that meet the specific binding criteria. Rather, additionally, or alternatively, the techniques developed by the inventors can be applied to predict whether a protein has any suitable characteristic, and to design proteins having the characteristic.

In some embodiments, this may be achieved using the same pre-trained language model used to train the binding affinity prediction model. This is because the pre-trained language model is trained to encode a wide variety and large volume of amino acid sequences and is not specific to a target or candidate protein. To adapt the model to a different application, the pre-trained language model may be fine-tuned according to embodiments of the technology described herein, using training data specific to the application. For example, to predict hydrophobicity, the pre-trained language model may be fine-tuned, according to the techniques described herein, using training data that includes amino acid sequences and measured hydrophobicity of proteins having the amino acid sequences.

Accordingly, embodiments of the technology described herein may be adapted to design proteins optimized for a variety of applications including, for example, binding, stability, manufacturability, or any other suitable applications.

### EXAMPLES

Following below is an example of using embodiments of the technology described herein to design libraries of single-chain variable fragments (scFvs) that were then empirically measured. Experiments were performed to show that antibodies designed according to the techniques developed by the inventors outperform antibodies designed according to conventional techniques. The example includes the following sections: “Overview,” “Results: ML-generated scFv libraries outperform conventional directed evolution,” “Results: ML-generated libraries can be highly diverse,” “Results: Model performance and sampling diversity are important factors in generating a quality library,” “Results: Bayesian-based approach provides insights prior to experimental testing,” “Methods: Training Data for Language Models,” “Methods: Training Language Models,” “Methods: Training Sequence-to-Affinity Models,” “Methods: ML Extrapolated Fitness Functions,” “Methods: Optimization Strategies,” “Methods: ML-optimized ScFv Libraries,” “Methods: Evolution Directed Libraries,” “Methods: Experimental Validation of Designed Sequences,” “Methods: T-Distributed Stochastic Neighbor Embedding (t-SNE),” and “Methods: Biophysical Property Calculation, Statistical Analysis of Libraries.”

### Overview

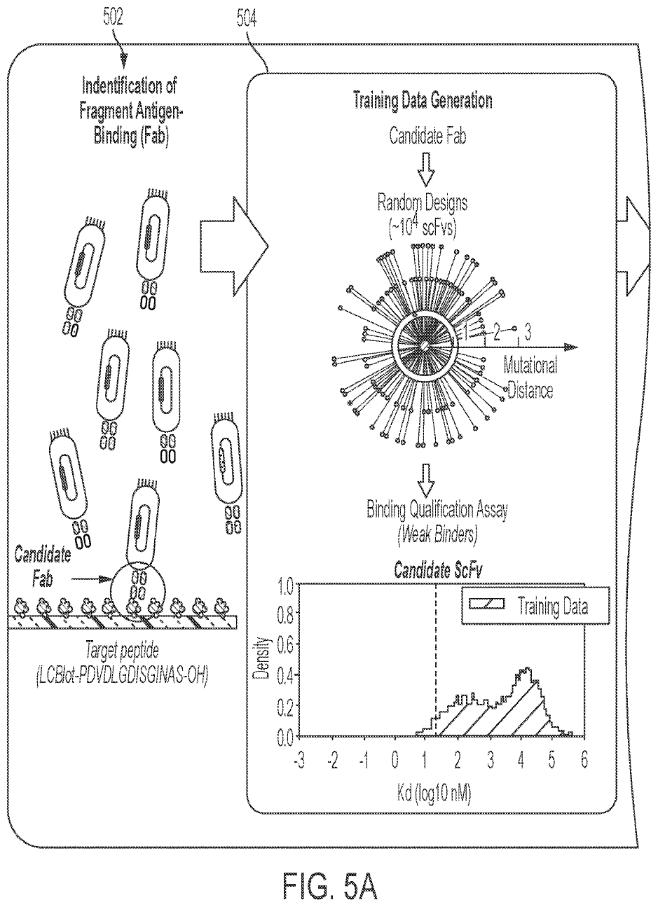

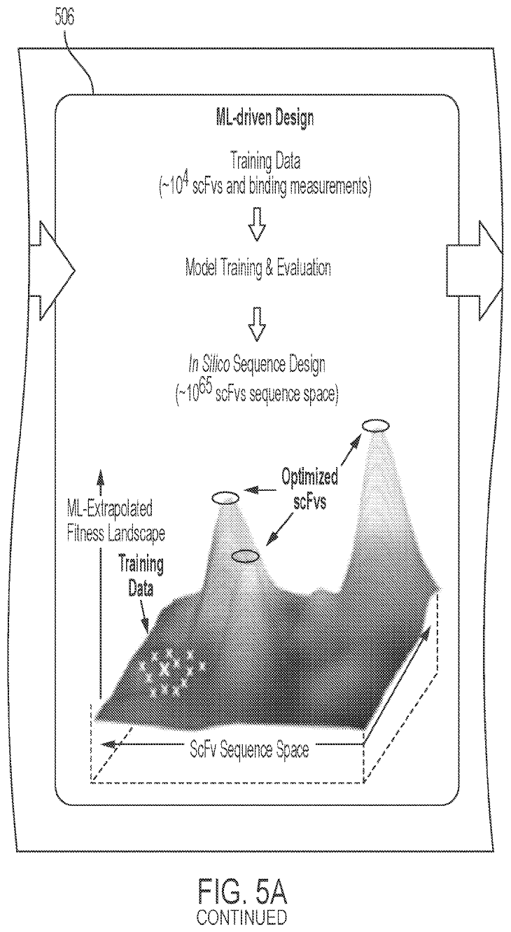

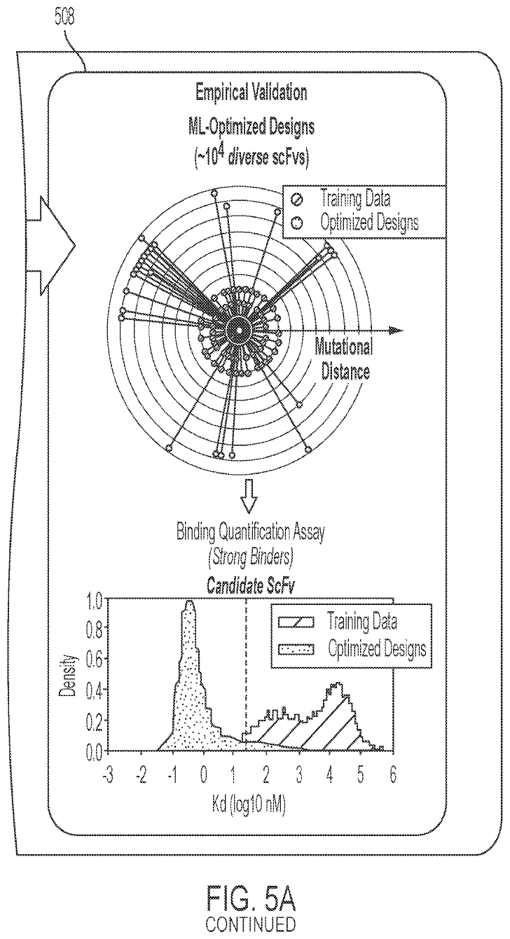

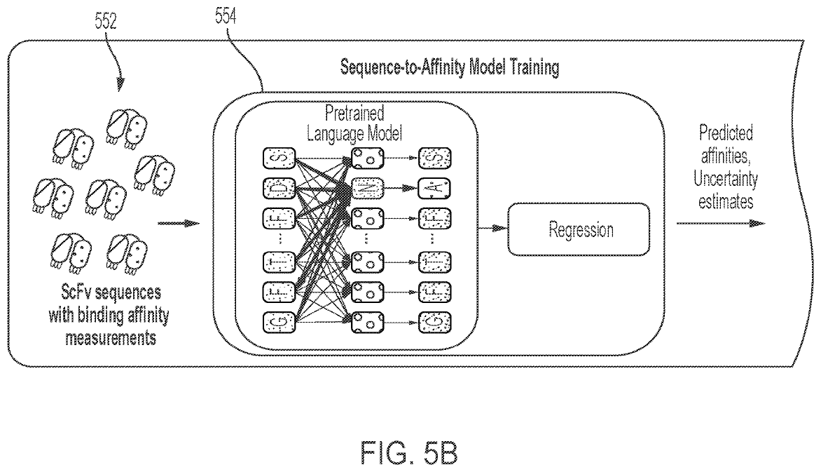

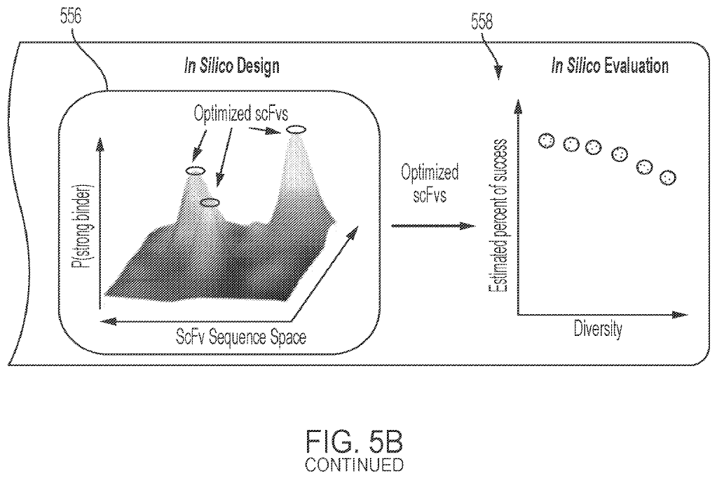

FIG. 5A and FIG. 5B depict an illustrative example of techniques used to engineer a candidate scFv against a target to generate high-affinity scFv libraries. At a high level, as shown in FIG. 5A, the techniques include (a) identifying a candidate Fab for a target at act 502; (b) high-throughput binding quantification of random mutants of the candidate scFv to the target to generate supervised training data at act 504; (b) machine learning-driven design to generate scFv libraries at act 506, and (d) empirical validation of designed libraries at act 508, providing a pool of potential scFv candidates for further development.

Supervised training data was generated using an engineered yeast mating assay. The target peptide is a conserved sequence found in the HR2 region of coronavirus spike proteins and to which neutralizing antibodies were previously identified. At act 502, a phage display campaign with a phage library containing naïve human Fabs was used to identify candidate scFv sequences (Ab-14, Ab-91, and Ab-95) that bind weakly to the target. The identified candidate sequences are shown in Table 1.

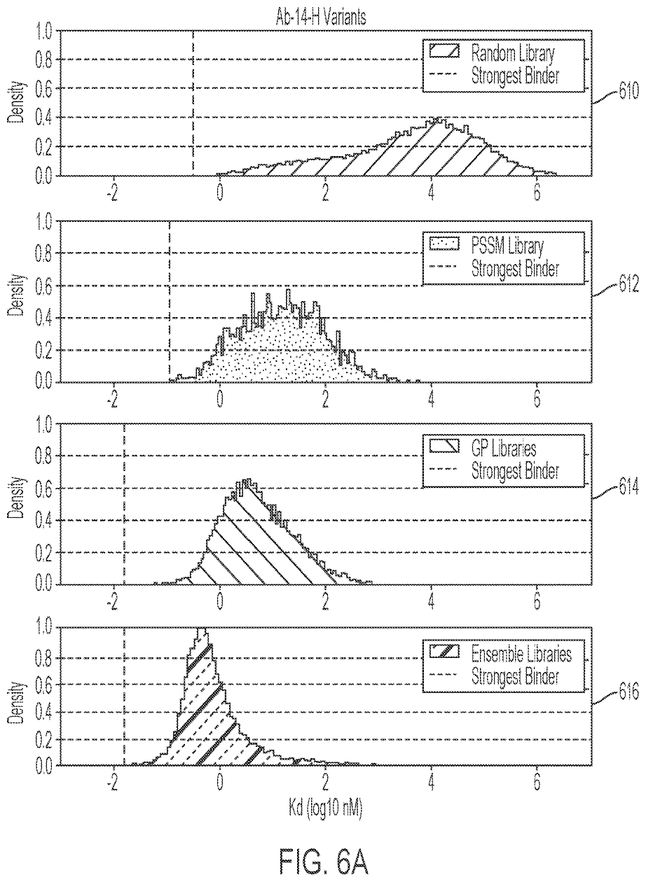

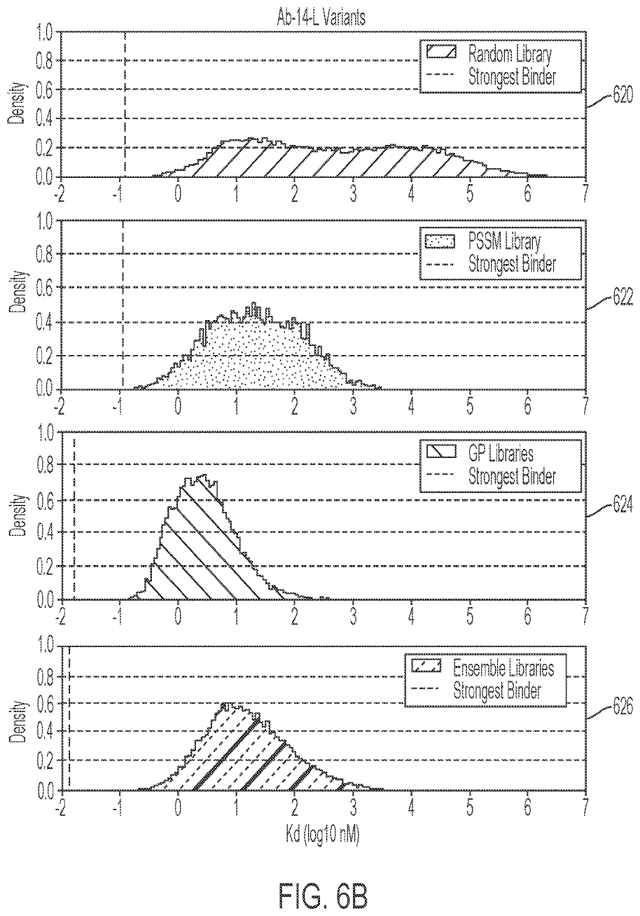

At act 504, the supervised training data was generated via random mutations of the candidate scFv along the entire CDR region, followed by high-throughput binding quantification to the selected target. All heavy and light chain sequences in the data were designed by performing random k=1, 2, 3 mutations within either the heavy chain or light chain CDRs of three candidate scFvs. Table 2 shows the distribution of mutations within each initial scFv library. Only the Ab-14 measurements (26,453 heavy chain, 26,223 light chain) were used as supervised training data for the sequence-to-affinity prediction. The binding measurements are provided on a log-scale, with lower values indicating stronger binding. FIGS. 6A-6B show the distribution of binding measurements. Average affinity was used for each sequence. For the training data (random library), the average value was computed for sequences with at least 1 (out of 3) empirically measure binding affinities. FIG. 6A shows the distributions of Ab-14 heavy-chain designs and the corresponding best binder. FIG. 6B shows the distributions of Ab-14 light-chain designs and the corresponding best binder. Plot 610 in FIG. 6A and plot 620 in FIG. 6B show the distribution of the training data (random library).

Examples of generating training data are described herein including at least in the section “Methods: Training Sequence-to-Affinity Models.”

At act 506, the training data combined with publicly available protein sequences was used to train, refine and evaluate machine learning models that drive the in silico sequence design process. An example implementation of act 506 is shown in FIG. 5B. At a high level, as shown in FIG. 5B, training, refining, and evaluating the machine learning models includes (a) supervised fine-tuning of pretrained language models on the training data 552 to predict binding affinities with uncertainty quantification at act 554, (b) construction of a Bayesian-based scFv fitness landscape extrapolated from the trained sequence-to-affinity model and in silico scFv design via Bayesian optimization at act 556, and (c) in silico design validation at act 558.

In more detail, four BERT masked language models were pre-trained, i.e., a protein language model, an antibody heavy chain model, an antibody light chain model and a paired heavy-light chain model. The protein language model was trained on the Pfam data, and antibody-specific language models were trained on human naïve antibodies from the Observed Antibody Space (OAS) database. Example techniques for pre-training language models are described herein including at least in the section “Methods: Training Language Models.” Data used for training such models are described herein including at least in the section “Methods: Training Data for Language Models.”

With respect to act 554 shown in FIG. 5B, two approaches were explored to train the sequence-to-affinity models: an ensemble method and Gaussian Process (GP). Both approaches use learned knowledge from pre-trained language models and provide meaningful sequence-to-affinity models from which high-affinity scFv libraries can be designed. Separate sequence-to-affinity models were trained for Ab-14-H heavy-chain variants and Ab-14-L light-chain variants using the corresponding training data.

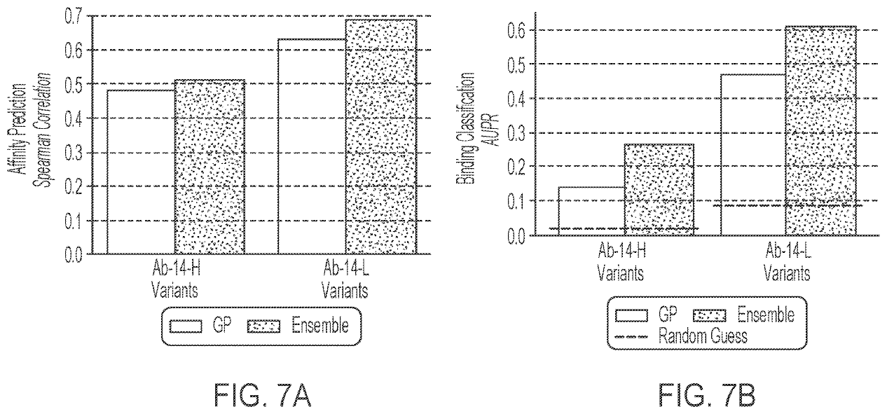

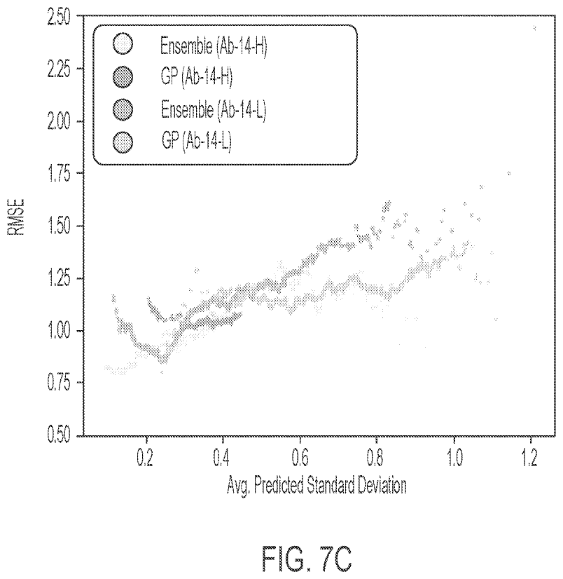

As shown in FIGS. 7A-7C, strong positive correlation was observed between predicted and experimentally measured binding affinities on the hold-out test data. FIG. 7A shows regression performance, and in particular that the ensemble model was more predictive than the GP model. With respect to FIG. 7B, an additional model evaluation task was defined: the model's ability to classify strong binders and weak binders. Sequences are labeled as strong binders if the empirically measured affinities are stronger (lower Kd values) than the initial candidate sequence and weak binders if the empirically measured affinities are weaker (higher Kd values) than the candidate sequence. The random guess computes the ratio of the number of strong binders to the total number of sequences in the hold-out test data. Area under the precision-recall curve (AUPR) is used to evaluate the binding classification task because it is tailored for the detection of rare events as suggested by random guess values. To compute the AUPR, all strong binders were labeled with a ground truth label ‘1’ and weak binders were labeled with a ground truth label ‘0’. The precision-recall (PR) curve was compute, from which the AUPR was calculated. The PR curve computes precision-recall pairs for different threshold values and the AUPR estimates the average precision of the model. FIG. 7C shows the relationship between the model's predictive uncertainty and model's prediction error captured by the root mean squared error (RMSE). For each predicted standard deviation in the hold-out test data, all test data with predicted standard deviations less than 0.05 away was found, and the corresponding averaged standard deviation and RMSE were computed. Positive correlation indicate that model's prediction tends to be less accurate when the prediction uncertainty is high. This overall trend is observed across all models. For ensemble models, model uncertainties capture the agreement between different regressors. Higher standard deviation indicates less agreement between regressors.

With respect to act 556, a Bayesian-based fitness landscape was constructed to map the entire scFv sequence to a posterior probability, i.e., the probability that the estimated binding affinity is better than the candidate scFv Ab-14, to generate high-affinity scFv libraries. This is in contrast to a fitness landscape that goes directly from sequence to estimated binding affinity. To perform optimization to maximize the posterior probability, the choice of sampling algorithm influences the library diversity. Three strategies were used: hill climb (HC), genetic algorithm (GA) and Gibbs sampling. HC is a greedy algorithm that performs a local search and only finds local maximums. GA is an evolutionary-based algorithm that is more robust in exploiting the sequence space further away from the initial sequence. Gibbs sampling takes sequential actions in a manner that balances exploitation and exploration and can generate sequences with high diversity.

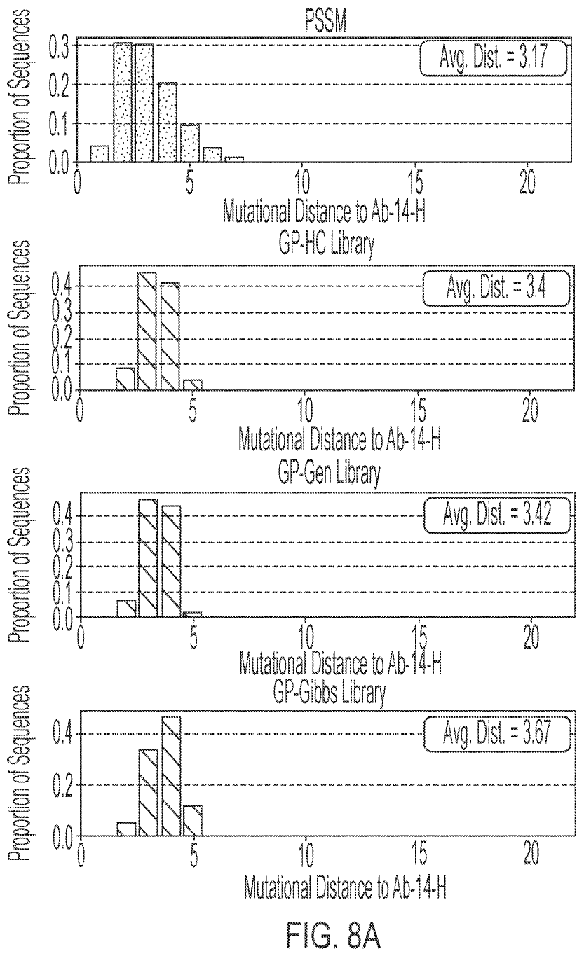

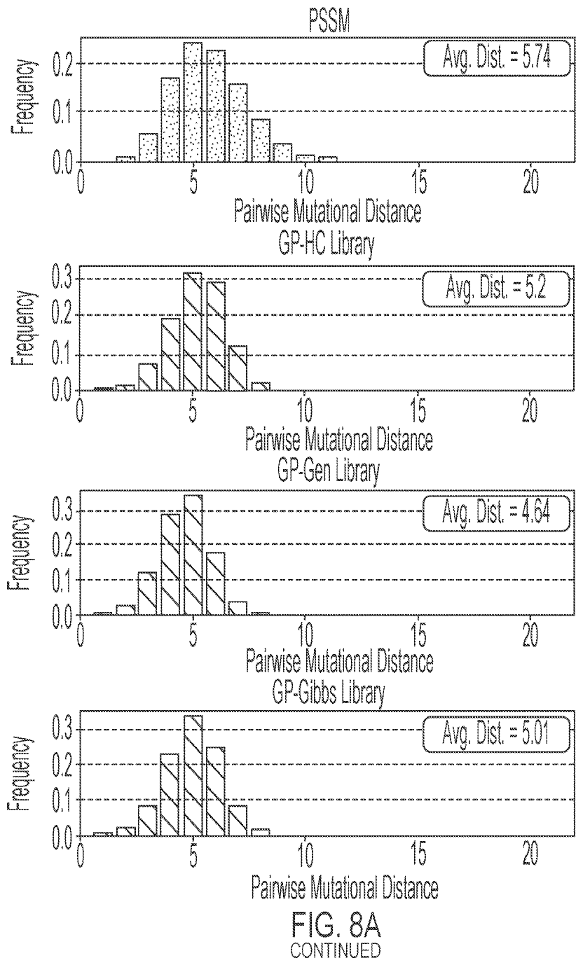

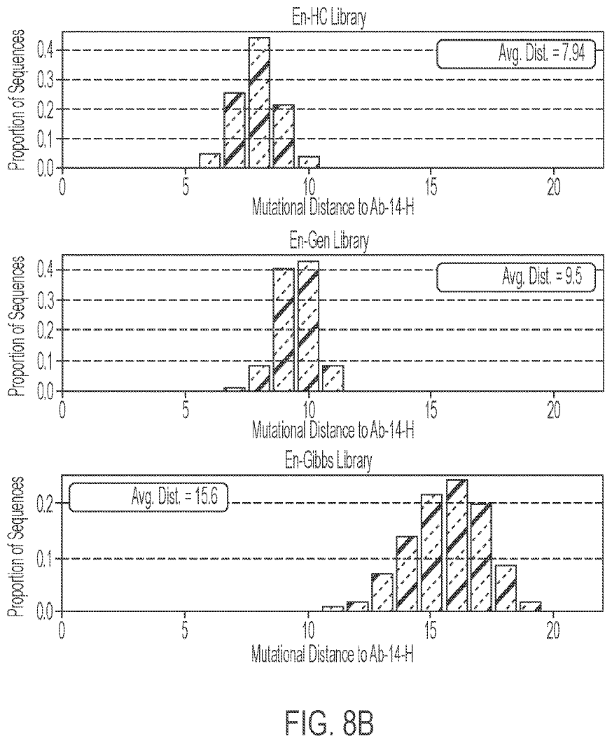

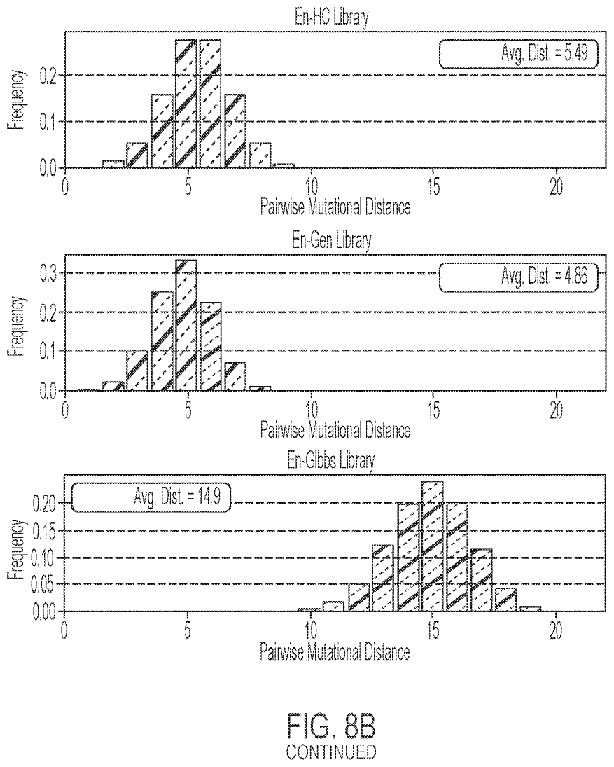

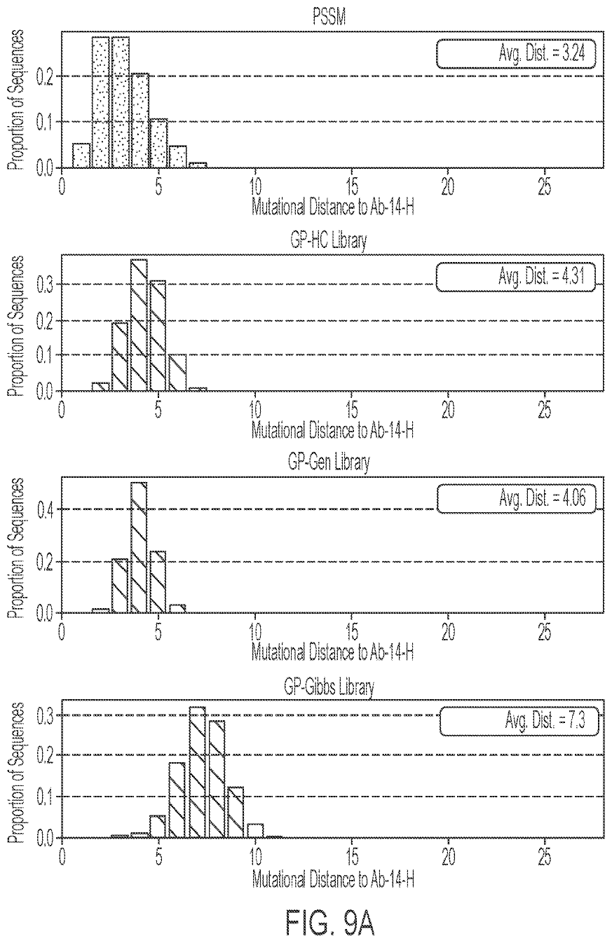

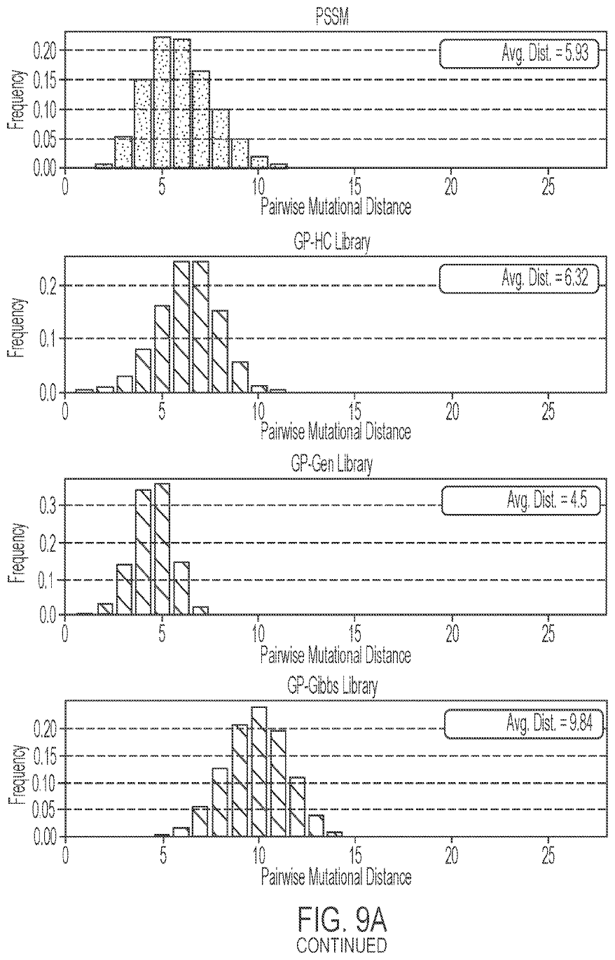

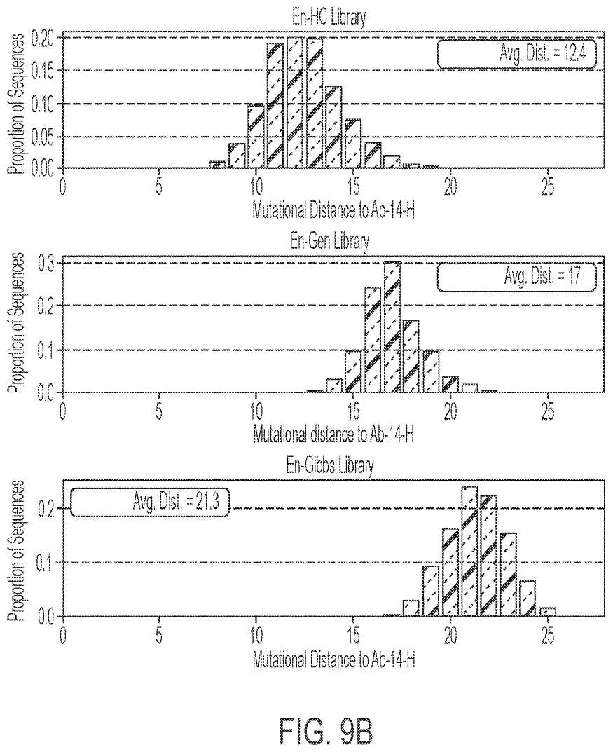

The sampling approaches were applied to generate heavy chain and light chain variant scFvs that optimize Ab-14. A Position-Specific Score Matrix (PSSM)-based method representative of conventional directed evolution approaches was also used to generate a control sequence set. The generated sequences from each method were rank-ordered based on the posterior probability and top sequences were selected. This resulted in seven scFv libraries per chain: three libraries from optimizing the ensemble-based fitness function (namely, En-HC, En-GA and En-Gibbs), three libraries from optimizing the Gaussian Process-based fitness function (namely, GP-HC, GP-GA, GP-Gibbs), and one PSSM library. scFv mutants were also generated with an average of k=2 random mutations from the 10 strongest binders of the supervised training data. FIGS. 8A-8B show the distribution of diversity metrics by library for Ab-14-H variants. FIGS. 9A-9B show the distribution of diversity metrics by library for Ab-14-L variants.

Returning to FIG. 5A, at act 508, the designed libraries were experimentally validated, providing thousands of potential antibody candidates for development. All sequences included in the seven libraries were synthesized and experimentally tested using the same high-throughput yeast display method as for the training data generation; Tables 3 and 4 provide the exact number of sequences from each library.

The empirical binding distribution of the training data was compared with the PSSM library and machine learning (ML)-designed sequences, as shown in FIGS. 6A and 6B. The ML designs were shown to be significantly stronger binders than the training data. Notably, more than 25% of ensemble-based Ab-14-H variant designs had stronger measured binding affinities than the strongest measured binder in the training data, whereas only 0.9% of PSSM-based Ab-14-H variant designs outperform the strongest measured binder in the training data. The ML-driven designs also produced highly diverse scFvs (sequences as far as 23 mutations away), with strong on-target binding (the best design is 28.7-fold better than the conventional directed evolution approach), and high success rate (as high as 99%).

TABLE 1
Target sequence and candidate scFv sequences (CDRs in bold).

| Target | PDVDLGDISGINAS |
|---|---|
| Candidate Chains | scFv Sequences |
| AB-14-H | EVQLVETGGGLVQPGGSLRLSCAASGFTLNSYGISWVRQAPGKGPEW |
| | VSVIYSDGRRTFYGDSVKGRFTISRDTSTNTVYLQMNSLRVEDTAVY |
| | YCAKGRAAGTFDSWGQGTLVTVSS |
| AB-14-L | DVVMTQSPESLAVSLGERATISCKSSQSVLYESRNKNSVAWYQQKAG |
| | QPPKLLIYWASTRESGVPDRFSGSGSGTDFTLTISSLQAEDAAVYYC |
| | QQYHRLPLSFGGGTKVEIK |
| AB-91-H | EVOLVESGGGLVQPGRSLRLSCAASGFTFDDYAMHWVRQAPGKGLE |
| | WVSGISWNSGSIGYADSVKGRFTISRDNAENSLYLQMNSLRAEDTALY |
| | YCAKVGRGGGYFDYWGQGTLVTVSS |
| AB-91-L | QAVLTQPSSLSASPGASVSLTCTLRSGINVGTYRIYWYQQKPGSPPQY |
| | LLRYKSDSDKQQGSGVPSRFSGSKDASANAGILLISGLQSEDEADYYC |
| | MIWHSSAWVFGGGTKLTVL |
| AB-95-H | EVQLVESGAEVKKPGASVKVSCKASGYTFTSYGISWVRQAPGQGLEW |
| | MGWISAYNGNTNYAQKLQGRVTMTTDTSTSTAYMELRSLRSDDTAV |
| | YYCARVGRGVIDHWGQGTLVTVSS |
| AB-95-L | SSELTQDPAVSVALGQTVRITCEGDSLRYYYANWYQQKPGQAPILVIY |
| | GKNNRPSGIADRFSGSNSGDTSSLIITGAQAEDEADYYCSSRDSSGFQV |
| | FFGAGTKLTVL |

TABLE 2
Distribution of mutations within each initial scFv library.
The numbers before and after the slash line represent the
number of variants present in the experimental measurements
and the total number of variant designs, respectively.

| Library | k = 1 | k = 2 | k = 3 |
|---|---|---|---|
| Ab-91-H Variants | 521/684 | 3,131/4,141 | 18,820/25,075 |
| Ab-14-H Variants | 594/665 | 3,671/4,089 | 22,188/25,146 |
| Ab-14-L Variants | 552/627 | 3,491/3,982 | 22,180/25,291 |
| Ab-95-L Variants | 548/551 | 3,743/3,755 | 25,526/25,594 |

TABLE 3
Percentage incorporation of Ab-14-H designs by library.

| Library | No. Sequences | No. Sequences | Overall % Present |
|---|---|---|---|
| Ensemble-HC | 6,000 | 5,344 | 89% |
| Ensemble-Gen | 6,000 | 5,310 | 89% |
| Ensemble-Gibbs | 6,000 | 4,879 | 81% |
| GP-HC | 6,000 | 5,152 | 86% |
| GP-Gen | 6,000 | 5,313 | 89% |
| GP-Gibbs | 6,000 | 5,284 | 88% |
| PSSM | 7,748 | 6,510 | 84% |

TABLE 4
Percentage incorporation of Ab-14-L designs by library.

| Library | No. Sequences | No. Sequences | Overall % Present |
|---|---|---|---|
| Ensemble-HC | 6,000 | 5,962 | 99% |
| Ensemble-Gen | 6,000 | 5,960 | 99% |
| Ensemble-Gibbs | 6,000 | 5,950 | 99% |
| GP-HC | 6,000 | 5,965 | 99% |
| GP-Gen | 6,000 | 5,989 | 100% |
| GP-Gibbs | 6,000 | 5,987 | 100% |
| PSSM | 8,257 | 8,188 | 99% |

Results: ML-Generated scFv Libraries Outperform Convention Directed Evolution.

The quality of each ML-derived scFv library was assessed by comparing the binding strength of the best design and the percent of success to the PSSM-generated library. The percent of success was defined as the percent of scFvs that have a better empirical binding score than the initial candidate scFv, Ab-14. PSSM libraries were chosen as comparators because they better reflect the traditional optimization process and are generally better than random mutation libraries.

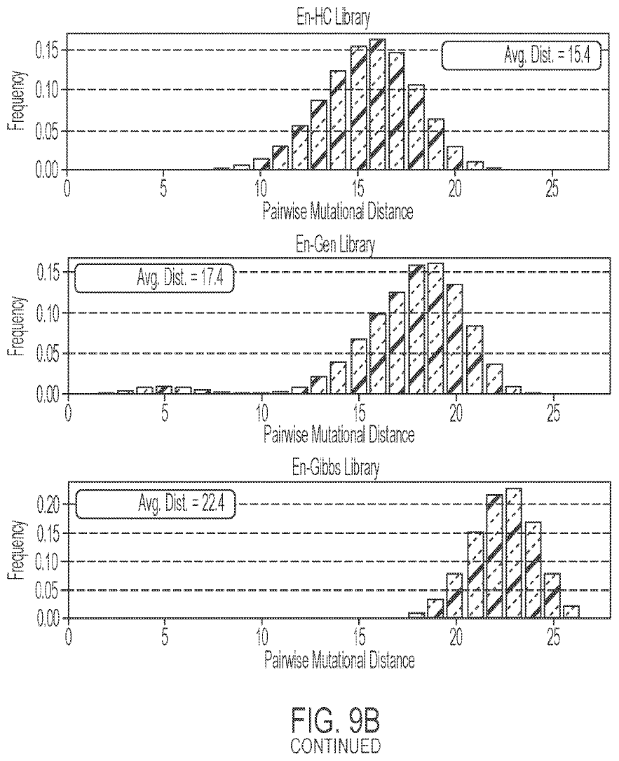

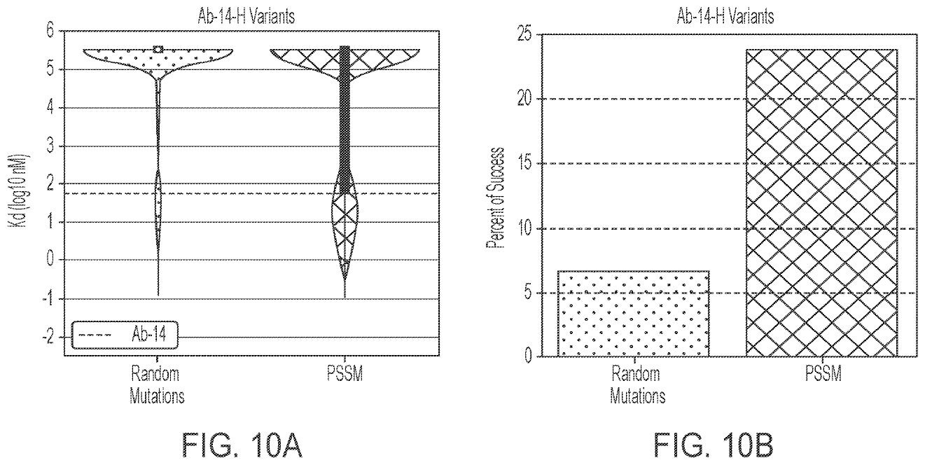

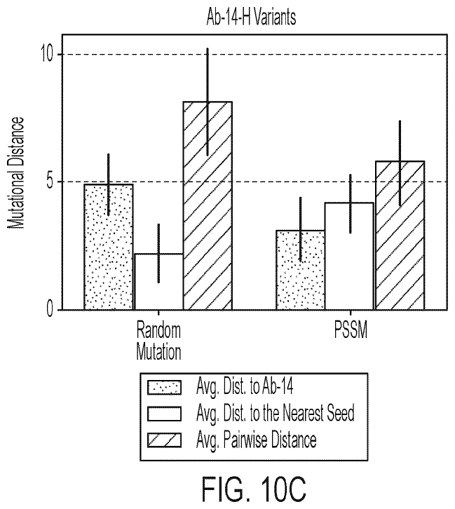

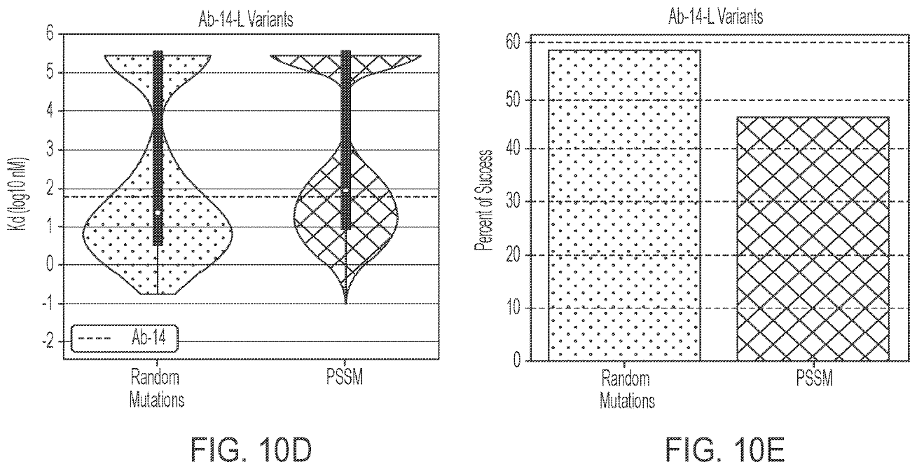

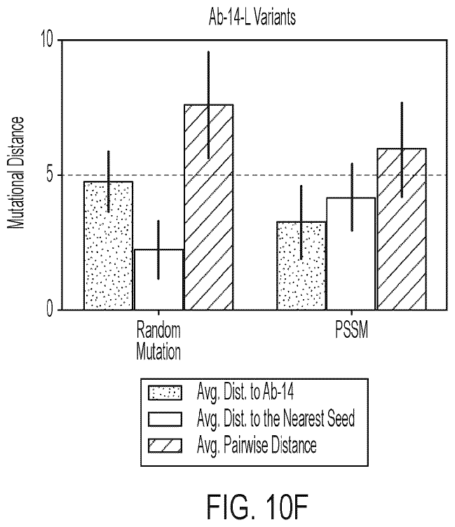

FIGS. 10A-10F show results of comparing the ML-generated scFv libraries to the PSSM libraries. For sequences having at least 3 (out of 6) empirical binding affinities, the averaged values were used as ground-truth measured affinities. All evaluations were performed over n=1616, 6510 Ab-14-H variant designs and n=465, 8188 Ab-14-L variant designs generated by random mutations and PSSM, respectively. The violin plot shown in FIG. 10A is used to depict summary statistics and empirically measured affinity distribution of Ab-14-H heavy chain designs (center: median; limits: 1st and 3rd quartile; whiskers: +/−1.5 IQR). Affinities of unsuccessful sequences were set to be 5.48 (the largest assay value of all Ab-14-H variants). FIG. 10B shows the percent of sequences that have stronger empirical binding affinity than the candidate antibody for all the Ab-14-H variant libraries. FIG. 10C is a diversity comparison for all the Ab-14-H variant libraries. Data are presented as mean values and +/−standard deviation. The average distance to the nearest seed sequences is added to the comparison because random mutations are generated by randomly mutating the seed sequences. The violin plot shown in FIG. 10D is used to depict summary statistics and empirically measured affinity distribution of Ab-14-L light chain designs (center: median; limits: 1st and 3rd quartile; whiskers: +/−1.5 IQR). Affinities of unsuccessful sequences were set to be 5.53 (the largest assay value of all Ab-14-L variants). FIG. 10E shows the percent of success for all Ab-14-L variant libraries. FIG. 10F is a diversity comparison for all the Ab-14-L variant libraries. Data are presented as mean values and +/−standard deviation. Table 5 contains characterization of the best binding scFv from each library. Sequences of these scFvs can be found in Tables 6 and 7.

As shown in FIGS. 10A-10F and in Tables 5, 6, and 7, the best scFvs from ML-optimized libraries are significantly stronger binders than those from the PSSM library, and generally have more mutations. The strongest binding heavy-chain design is from the En-Gen library and binds 28.7-fold stronger than the strongest scFv in the PSSM library. The best light-chain design is in the En-Gibbs library achieving a 7.7-fold improvement over the best scFv from the PSSM library. Note that the best heavy-chain scFv binds much stronger to the target than the best light-chain scFv. To investigate further, all designed scFvs were rank-ordered across different libraries by the empirically-measured binding affinity. As shown in FIG. 11, heavy-chain designs are generally stronger binders than light-chain designs.

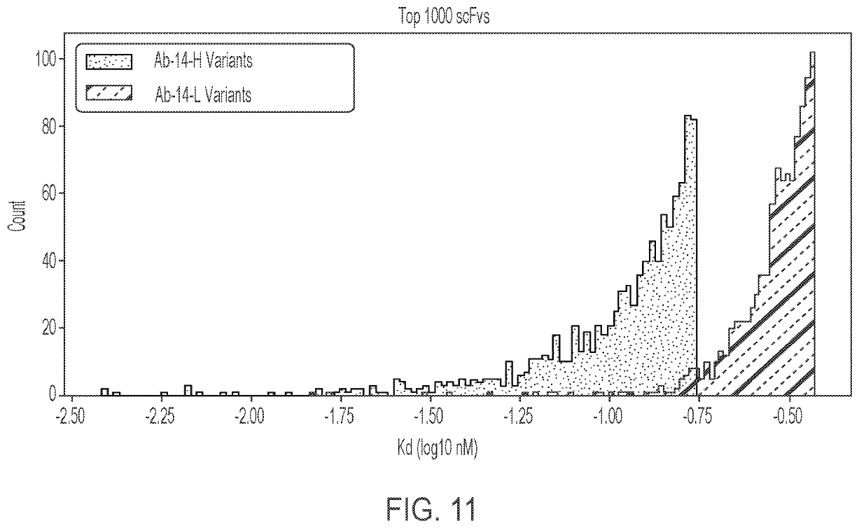

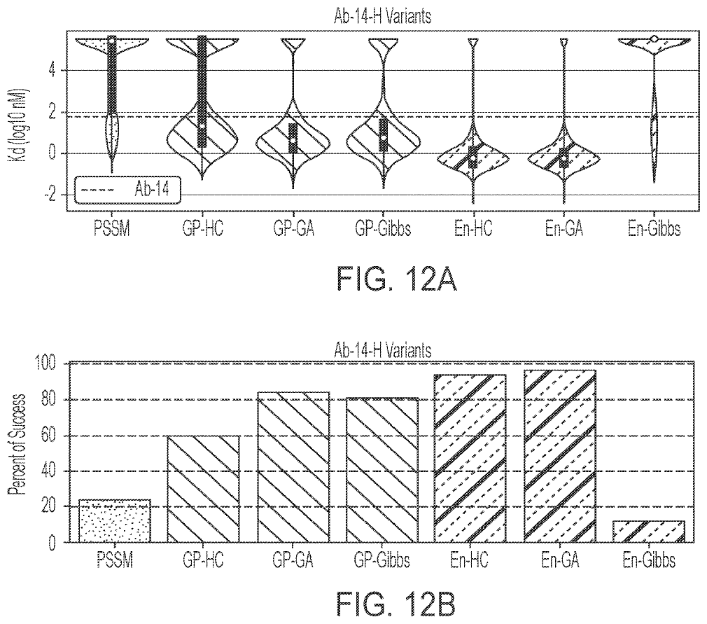

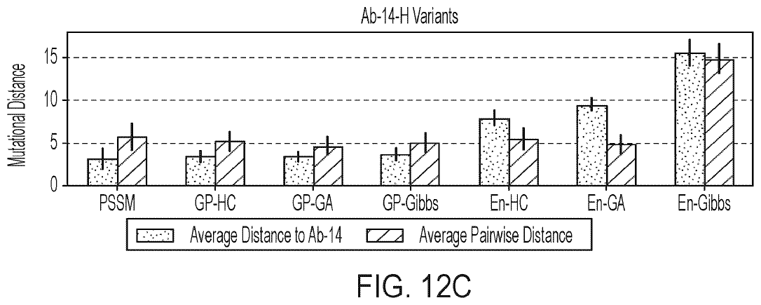

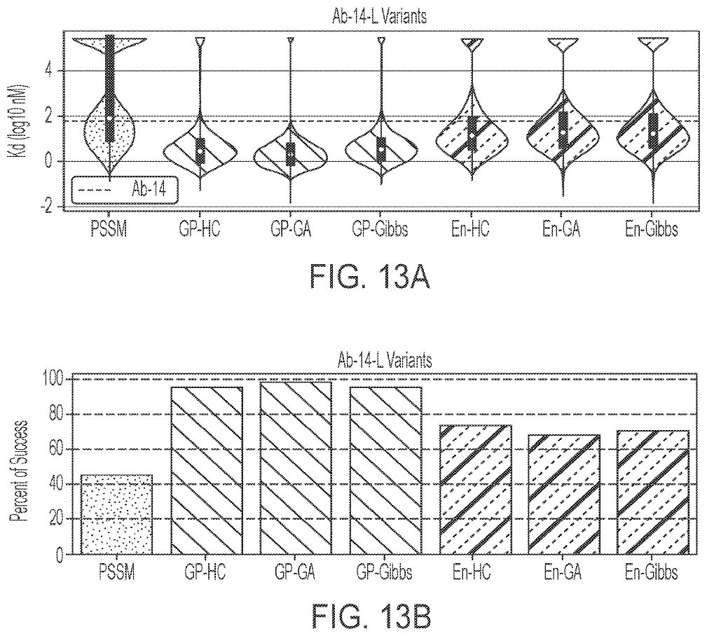

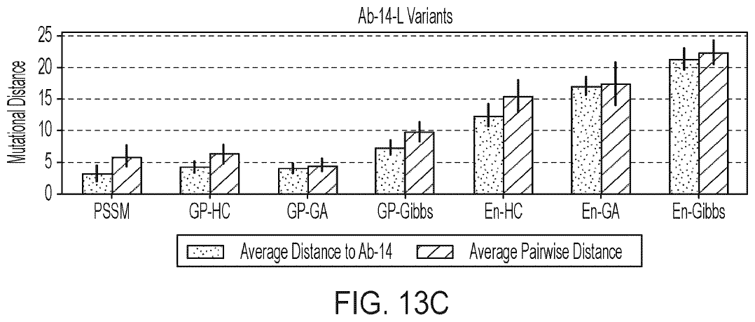

FIGS. 12A-12C and FIGS. 13A-13C shows the performance and diversity of designed libraries. For sequences with at least 3 (out of 6) empirical binding affinities, averaged values were used as the ground-truth. The rest of the sequences (with less than 3 empirical measurements) were considered as un-successful designs. All evaluations were performed over n=6510, 5152, 5313, 5284, 5344, 5310, 4879 Ab-14-H variant designs and n=8188, 5965, 5989, 5987, 5962, 5960, 5950 Ab-14-L variant designs generated by PSSM, GP-HC, GP-GA, GP-Gibbs, En-HC, En-GA and En-Gibbs, respectively (as shown in Tables 3 and 4), where GP and En denote Gaussian Process and Ensemble models, and HC, GA and Gibbs denote hill climb, genetic and Gibbs sampling algorithms, respectively.

FIG. 12A is a violin plot used to depict summary statistics and empirically measured affinity distribution of Ab-14-H heavy chain designs (center: median; limits: 1st and 3rd quartile; whiskers: +/−1.5 IQR). Affinities of unsuccessful sequences are set to be 5.48 (the largest assay value of all Ab-14-H variants). As shown in FIG. 12A, with the exception of sequences in the En-Gibbs library, all ML-optimized libraries outperform the PSSM library in terms of median binding affinity.

FIG. 12B shows the percent of sequences that have stronger empirical binding affinity than the candidate antibody for all the Ab-14-H variant libraries. As shown in FIG. 12B, with the exception of sequences in the En-Gibbs library, all ML-optimized libraries are significantly more successful than the 23.8% success of the PSSM library.

FIG. 13A is a violin plot used to depict summary statistics and empirically measured affinity distribution of Ab-14-L light chain designs (center: median; limits: 1st and 3rd quartile; whiskers: +/−1.5 IQR). Affinities of unsuccessful sequences are set to be 5.53 (the largest assay value of all Ab-14-L variants). As shown in FIG. 13A, all ML-optimized libraries outperform the PSSM library in terms of median binding.

FIG. 13B shows the percent of success for all the Ab-14-L variant libraries. As shown in FIG. 13B, all ML-optimized libraries outperform the PSSM library in percent of success whereas the PSSM library is 45.6% successful. The percent of success of GP-based libraries (95.7-99%) further outperforms all ensemble-based libraries (67.9-73.5%).

TABLE 5
Characterization of the top scFv from each library.

| | Best Ab-14-H Variant Design | | | Best Ab-14-L Variant Design | | |
|---|---|---|---|---|---|---|
| | Predicted | Mutational | Fold | Predicted | Mutational | Fold |
| | Affinity | Distance | Improvement | Affinity | Distance | Improvement |
| Library | (pM) | to Ab-14 | Over PSSM | (pM) | to Ab-14 | Over PSSM |
| PSSM | 109.602 | 4 | 1.0 | 113.053 | 3 | 1.0 |
| GP-HC | 52.179 | 3 | 2.1 | 57.944 | 3 | 2.0 |
| GP-GA | 20.483 | 4 | 5.4 | 16.454 | 3 | 6.9 |
| GP-Gibbs | 15.541 | 4 | 7.1 | 98.980 | 9 | 1.1 |
| En-HC | 3.817 | 7 | 28.7 | 156.090 | 11 | 0.7 |
| En-GA | 3.923 | 10 | 27.9 | 30.400 | 17 | 3.7 |
| En-Gibbs | 38.126 | 15 | 2.9 | 14.608 | 23 | 7.7 |

TABLE 6
The best heavy chain by library (CDRs in bold).

| Libraries | Best Ab-14-H Variant |
|---|---|
| Random | EVQLVETGGGLVQPGGSLRLSCAASGFTLNQYGISWVRQAPGKGPEW |
| Mutations | VSVIYSDGIRTFYSDSVKGRFTISRDTSTNTVYLQMNSLRVEDTAVYY |
| | CAKGRAAPFFDSWGQGTLVTVSS |
| PSSM | EVQLVETGGGLVQPGGSLRLSCAASGFTLNEYGISWVRQAPGKGPEW |
| | VSVIYADGRRTFYADSVKGRFTISRDTSTNTVYLQMNSLRVEDTAVY |
| | YCAKGRAAGTFDVWGQGTLVTVSS |
| GP-HC | EVQLVETGGGLVQPGGSLRLSCAASGFTLNEYGISWVRQAPGKGPEW |
| | VSVIYSDGRRTFYSDSVKGRFTISRDTSTNTVYLQMNSLRVEDTAVYY |
| | CAKGRAAGTFDIWGQGTLVTVSS |
| GP-Gen | EVQLVETGGGLVQPGGSLRLSCAASGFSLNEYGISWVRQAPGKGPEW |
| | VSVIYSDGRRTFYGDSVKGRFTISRDTSTNTVYLQMNSLRVEDTAVYY |
| | CAKGQAAGTFDFWGQGTLVTVSS |
| GP-Gibbs | EVQLVETGGGLVQPGGSLRLSCAASGFSLNEYGISWVRQAPGKGPEW |
| | VSVIYSDGRRTFYGDSVKGRFTISRDTSTNTVYLQMNSLRVEDTAVYY |
| | CAKGNAAGTFDQWGQGTLVTVSS |
| En-HC | EVQLVETGGGLVQPGGSLRLSCAASGFDLNEYGISWVRQAPGKGPEW |
| | VSVIYADGRRTFYTDSVKGRFTISRDTSTNTVYLQMNSLRVEDTAVYY |
| | CAKGEVAGTFDGWGQGTLVTVSS |
| En-Gen | EVQLVETGGGLVQPGGSLRLSCAASGFDLNEYGISWVRQAPGKGPEW |
| | VSVIYADGSRKAYADSVKGRFTISRDTSTNTVYLQMNSLRVEDTAVY |
| | YCAKGNNAGTFDVWGQGTLVTVSS |
| En-Gibbs | EVQLVETGGGLVQPGGSLRLSCAASEFDIQEYGISWVRQAPGKGPEW |
| | VSVIYADGKREAYKDKFKGRFTISRDTSTNTVYLQMNSLRVEDTAVY |
| | YCAKGQVAGTFDAWGQGTLVTVSS |

TABLE 7
The best light chain by library (CDRs in bold).

| Libraries | Best Ab-14-H Variant |
|---|---|
| Random | DVVMTQSPESLAVSLGERATISCKSSQSVLYESRNKNSVAWYQQKAG |
| Mutations | QPPKLLIYWASTRESGVPDRFSGSGSGTDFTLTISSLQAEDAAVYYCQQ |
| | YHRLPLSFGGGTKVEIKDVVMTQSPESLAVSLGERATISCKQSQEVLFE |
| | SRNKNSVAWYQQKAGQPPKLLIYDASTRESGVPDRFSGSGSGTDFTLT |
| | ISSLQAEDAAVYYCQQYHRLPLSFGGGTKVEIK |
| PSSM | DVVMTQSPESLAVSLGERATISCKLSQSVLYESRNKNSVAWYQQKAG |
| | QPPKLLIYDASLRESGVPDRFSGSGSGTDFTLTISSLQAEDAAVYYCQQ |
| | YHRLPLSFGGGTKVEIK |
| GP-HC | DVVMTQSPESLAVSLGERATISCKSSQSVLYESGNKNSVAWYQQKAG |
| | QPPKLLIYDASTREDGVPDRFSGSGSGTDFTLTISSLQAEDAAVYYCQQ |
| | YHRLPLSFGGGTKVEIK |
| GP-Gen | DVVMTQSPESLAVSLGERATISCKVQQSVLYESRNKNSVAWYQQKA |
| | GQPPKLLIYGASTRESGVPDRFSGSGSGTDFTLTISSLQAEDAAVYYCQ |
| | QYHRLPLSFGGGTKVEIK |
| GP-Gibbs | DVVMTQSPESLAVSLGERATISCKLMQEDEYQSRNPNSVAWYQQKA |
| | GQPPKLLIYHASERESGVPDRFSGSGSGTDFTLTISSLQAEDAAVYYCQ |
| | QYHRLPLSFGGGTKVEIK |
| En-HC | DVVMTQSPESLAVSLGERATISCMISESVMYESRNRNNVAWYQQKAG |
| | QPPKLLIYDHSTREDGVPDRFSGSGSGTDFTLTISSLQAEDAAVYYCQC |
| | YDRLPLSFGGGTKVEIK |
| En-Gen | DVVMTQSPESLAVSLGERATISCQISGIQGHMSTIKNNVAWYQQKAG |
| | QPPKLLIYEMVTRANGVPDRFSGSGSGTDFTLTISSLQAEDAAVYYCQ |
| | QYERLPLSFGGGTKVEIK |
| En-Gibbs | DVVMTQSPESLAVSLGERATISCNMVEDEAGDQKNSGNIAWYQQKA |
| | GQPPKLLIYSVDQREDGVPDRFSGSGSGTDFTLTISSLQAEDAAVYYCQ |
| | QYQKLPLMFGGGTKVEIK |

### Results: ML-Generated Libraries can be Highly Diverse.

Library diversity was measured using two mutational distance metrics:

$${d}_{a v g}^{A b - 14}$$

(the average distance to the initial Ab-14), and d_{pw} (the average pairwise distance). The former

$${d}_{a v g}^{A b - 14}$$

indicates how far the designs are from the training data and the latter d_{pw} measures the intra-library diversity.

FIG. 12C shows a diversity comparison for all Ab-14-H variant libraries. Data are presented as mean values and +/−standard deviation to show mutational variability of designed sequences from the initial candidate scFv. As shown in FIG. 12C, all ML-optimized libraries have higher

$${d}_{a v g}^{A b - 14}$$

than the PSSM library (with

$${d}_{a v g}^{A b - 14} = 3.1 ) .$$

The ensemble-based libraries also have significantly higher

$${d}_{a v g}^{A b - 14}$$

(7.9-15.6) than the GP-based libraries (3.4-3.7), indicating that the methods are able to extrapolate and design sequences that are far beyond the training data. In particular, sequences in the En-Gibbs library are on average 15.6 distance away from Ab-14-H and 14.9 distance away from each other.

FIG. 13C shows a diversity comparison for all the Ab-14-L variant libraries. Data are presented as mean values and +/−standard deviation. As shown in FIG. 13C, all ML-optimized libraries are significantly further away from Ab-14-L than the PSSM library, with

$${d}_{a v g}^{A b - 14} == 3.2$$

for the PSSM library,

$${d}_{a v g}^{A b - 14}$$

ranging from 4.5 to 7.4 for GP-based libraries and

$${d}_{a v g}^{A b - 14}$$

ranging from 12.4 to 21.3 for ensemble-based libraries. With the exception of GP-GA (d_{pw}=4.5), all ML-optimized libraries have higher d_{pw} (ranging from 6.3 to 22.4) than the PSSM library (d_{pw}=5.9). In particular, the En-Gibbs light-chain library includes sequences that are on average 21.3 distance away from Ab-14-L and 22.4 distance away from each other.

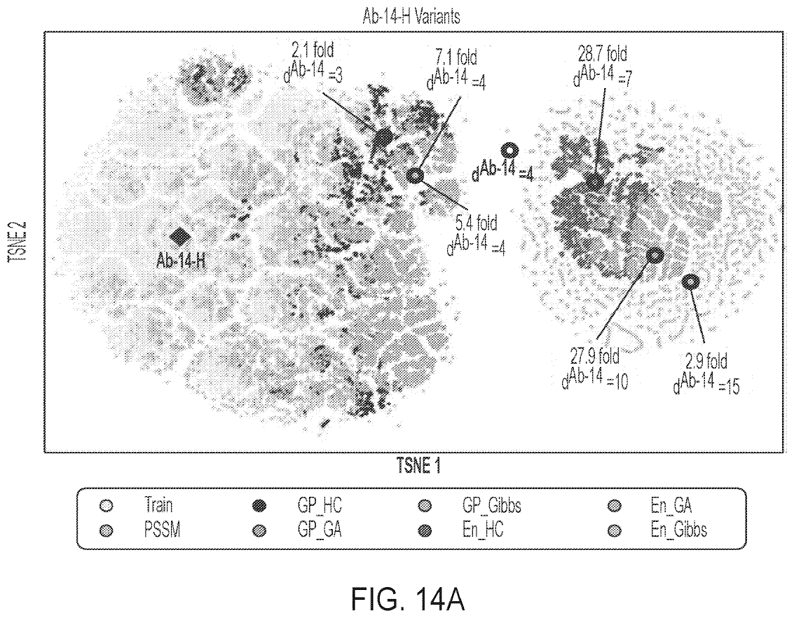

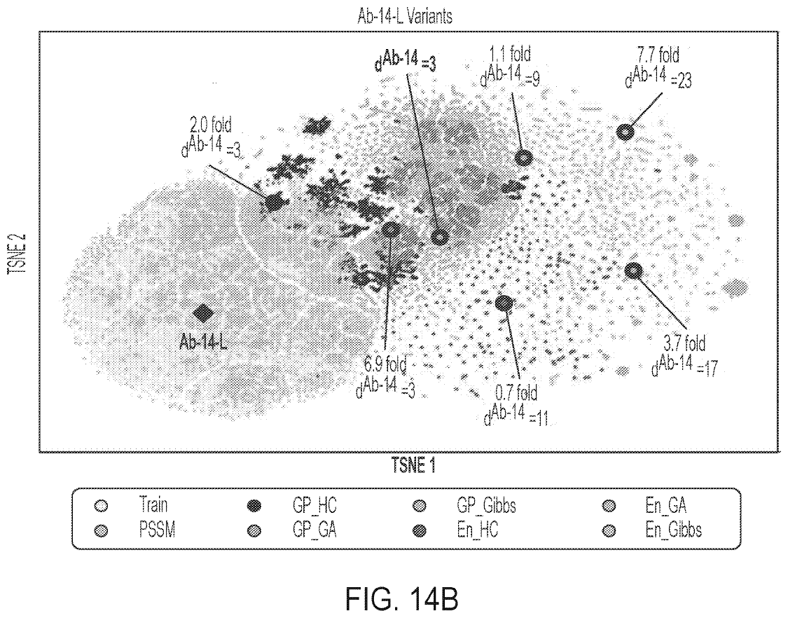

FIGS. 14A-14B show the 2-D embeddings of all scFv libraries and the training data. The t-SNE embeddings allow visualization of the sequence space by embedding sequences onto 2-D space, while approximately preserving the edit distance between sequences. GP and En denote Gaussian Process and Ensemble models, and HC, GA and Gibbs denote hill climb, genetic and Gibbs sampling algorithms, respectively. FIG. 14A shows 2-D embeddings of Ab-14-H variants. FIG. 14B shows 2-D embeddings of Ab-14-L variants. The initial candidate sequence Ab-14 is marked with a diamond marker. The best scFv variants from each library are marked in circles. The best ML-generated scFvs are labeled with fold improvement over the best PSSM scFv and the mutational distance from the candidate Ab-14 scFv. The best PSSM scFv is labeled with mutational distance only. Source data are provided as a Source Data file.

A similar trend was observed for both light- and heavy-chain designs, that is, the PSSM library is the closest to the training data while the ensemble-based libraries are the farthest away from the training data. Additionally, all optimization-based libraries occupy a distinct subspace from the training data and PSSM library, highlighting the extrapolating power of the various optimization approaches that were applied. Ensemble-based libraries are highly divergent and also group distinctly from the other libraries; both the best heavy- and light-chain designs were discovered via optimizing the ensemble-extrapolated fitness function, underlining the value of exploring further away from the initial candidate sequence.

### Results: Model Performance and Sampling Diversity are Important Factors in Generating a Quality Library.

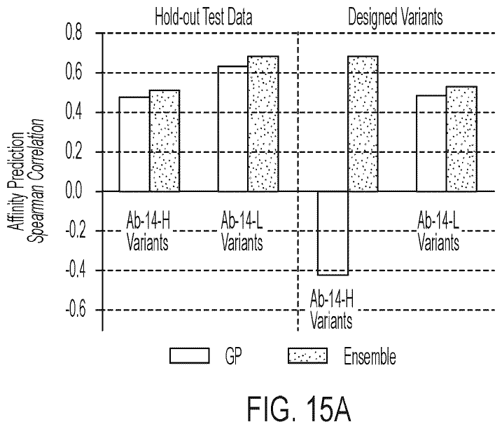

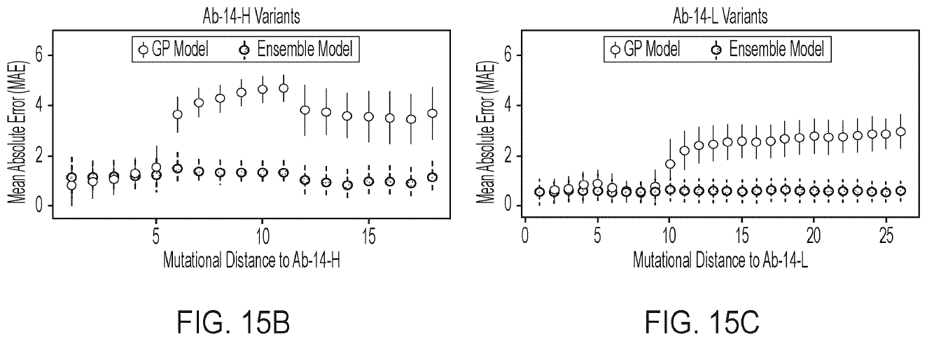

To understand important factors that determine the quality of a generated library, the performance of the two sequence-to-affinity models was evaluated, using held-out test data and empirical binding measurements of the designed sequences. FIGS. 15A-15C show the results of this evaluation. All evaluations were performed on sequences with at least 3 (out of 6) empirical binding affinities and the averaged values were used as the ground-truth. GP denotes Gaussian Process.

FIG. 15A shows the regression performance on hold-out test data and on the designed libraries; the ensemble model is more predictive than the GP model on both datasets. The Spearman correlation and the mean absolute error (MAE) of model predictions and measured values were compared. The ensemble sequence-to-affinity model was observed to do better at predicting affinity than the GP model. When evaluated on the held-out test data, Spearman correlation scores of both heavy- and light-chain ensemble models are slightly higher (heavy-chain model: 0.51; light-chain model: 0.69) than the respective GP models, as shown in FIG. 15A. When evaluated on designed Ab-14-L variants, the light-chain ensemble model was also slightly better. The most notable difference is when evaluating on designed Ab-14-H variants, where the heavy-chain ensemble model has a Spearman correlation of 0.69 but the heavy-chain GP model performs significantly worse (−0.42). This is primarily due to the prediction limit of the GP model on sequences that are far beyond the training data.

FIG. 15B and FIG. 15C show the performance of GP and ensemble models with respect to mutational distance from Ab-14. Data are presented in mean absolute error (MAE) and +/−SEM. The sample sizes of Ab-14-H variants for mutational distances ranging from 1 to 18 are n=93, 1337, 4485, 5009, 834, 296, 1316, 2400, 2855, 2304, 418, 53, 131, 148, 152, 124, 71, 33, respectively. The sample sizes of Ab-14-L variants for mutational distances ranging from 1 to 26 are n=258, 1784, 3696, 6287, 4168, 2097, 2037, 1748, 932, 675, 1042, 1095, 1109, 888, 933, 1447, 1632, 1090, 1025, 1063, 1317, 1168, 741, 341, 86, 18, respectively. As shown in FIGS. 15B-15C, ensemble models are more robust at extrapolating mutationally distant scFvs while the GP models do not predict well on sequences that are mutationally far away from Ab-14. The error bar of the heavy-chain ensemble model shows a non-trivial increase on sequences that are twelve or more mutations away from Ab-14, suggesting that the model's predictability decreases with increase in mutational distance. A sharp increase in MAE was observed on sequences with six or more mutations away from Ab-14-H for the heavy-chain GP model, and on sequences with ten or more mutations away from Ab-14-L for the light-chain GP model. Ensemble models exhibit no notable increase in MAE as the mutational distance increases, indicating that the ensemble approach is more generalizable to higher-order mutants than the GP model. Nevertheless, GP-based libraries, when compared to the PSSM library, are significantly more successful while having comparable sequence diversity as shown in FIGS. 12A-12C and FIGS. 13A-13C.

While ML-guided exploration of sequence space allows for identification of more scFvs with optimized binding, it is likely that if this set comes from diverse sequence space, it will also have diverse development properties thus limiting the chance of correlated downstream development failure. The choice of sampling algorithm may also be important to generate a diverse library with high affinity. When using the ensemble-extrapolated fitness landscape to engineer 14-Ab-H, hill climb and genetic algorithms found scFvs with significant (28.7 and 27.9-fold, respectively) increases in binding over the best PSSM-sampled scFv, as shown in Table 5, and both methods were highly successful (94.3% and 96% success, respectively), as shown in FIGS. 12A and 12B. However, when combined with the Gibbs sampling algorithm, the best scFv sampled was only 2.9-fold better, and shown in Table 5, and the library was generally unsuccessful. With the diversity metrics of the En-Gibbs-generated sequences almost double that of the En-HC and En-GA libraries, it indicates that the significant increase in diversity of the En-Gibbs library may have a detrimental effect on library affinity due to the eventual limit of the model predictability on sequences that are deemed too far from the training data, as shown in FIG. 12C. Interestingly, when engineering the light chain (14-Ab-L), the En-Gibbs combination found the strongest binder (7.7-fold improvement over PSSM) with a striking 23 mutations from the Ab-14-L sequence, as shown in Table 5. For the ensemble-based libraries, as the library diversity increased, so too did the binding strength of its top scFv, as shown in FIG. 13C and Table 5. En-HC, the least diverse ensemble-generated 14-Ab-L library, was the only library that failed to contain an scFv outperforming the top PSSM-generated scFv, as shown in Table 5. In this instance, the increased library diversity was beneficial, suggesting the value in exploring away from the initial candidate sequence. Hence, to avoid unsuccessful library designs while still being able to explore sufficiently high orders of mutants, it may be beneficial to control the diversity of sampled sequences via parameter tuning of the sampling algorithm and have the ability to explore the tradeoff between performance and diversity in silico prior to experimental testing.

### Results: Bayesian-Based Approach Provides Insights Prior to Experimental Testing.

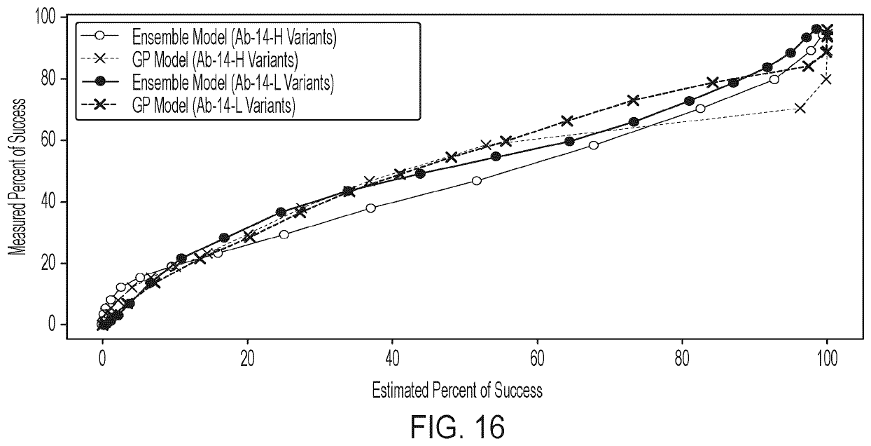

An in silico performance metric was defined that quantifies the binding performance of a library prior to experimental testing. With the Bayesian approach, the fitness score is the posterior probability of a sequence in the library having a stronger binding affinity than the candidate scFv Ab-14. The individual fitness scores of the full library were averaged to come up with a metric—an estimate of the probability of success (i.e., the estimated percent of sequences having a better binding performance than the threshold value). The utility of the metric was evaluated on the hold-out test data from the training scFv library as the threshold value that defines strong binders was varied. The results, shown in FIG. 16, show that the estimated percent of success matches well to the actual percent of success.

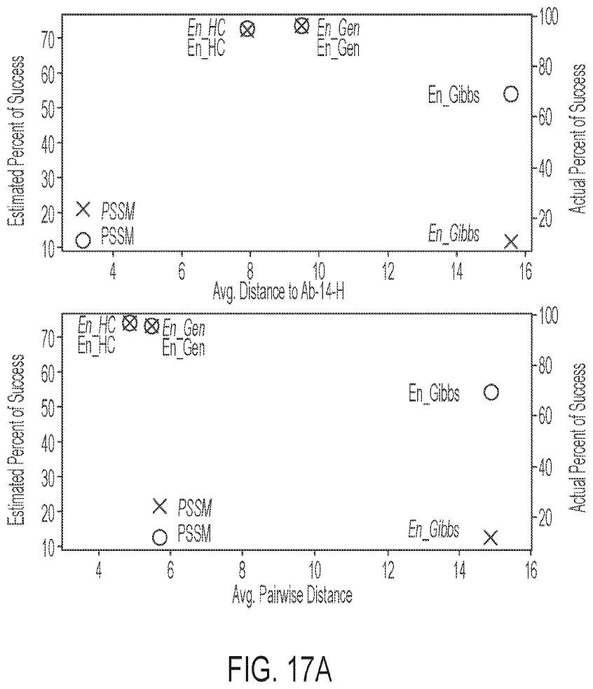

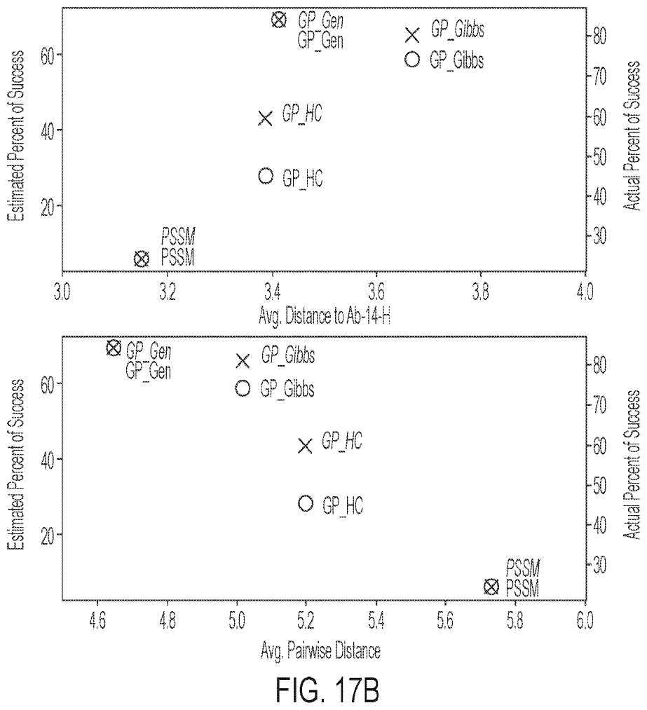

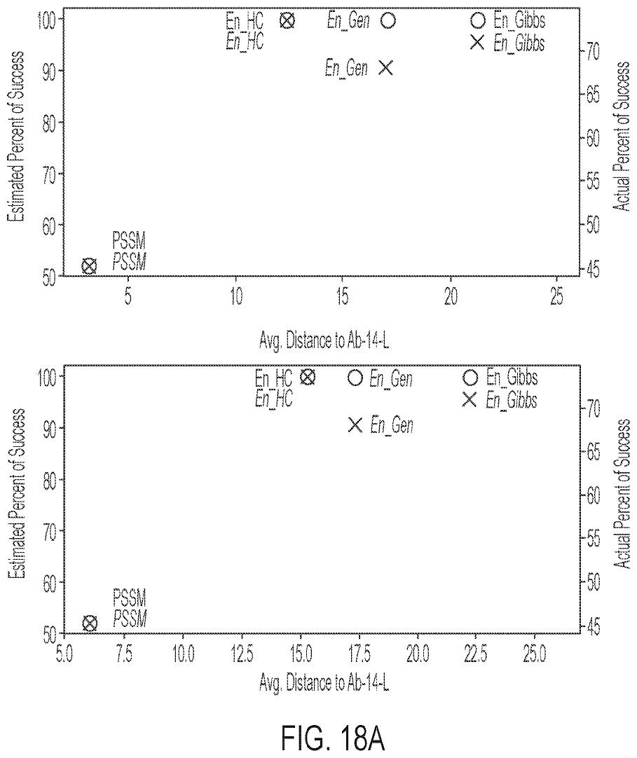

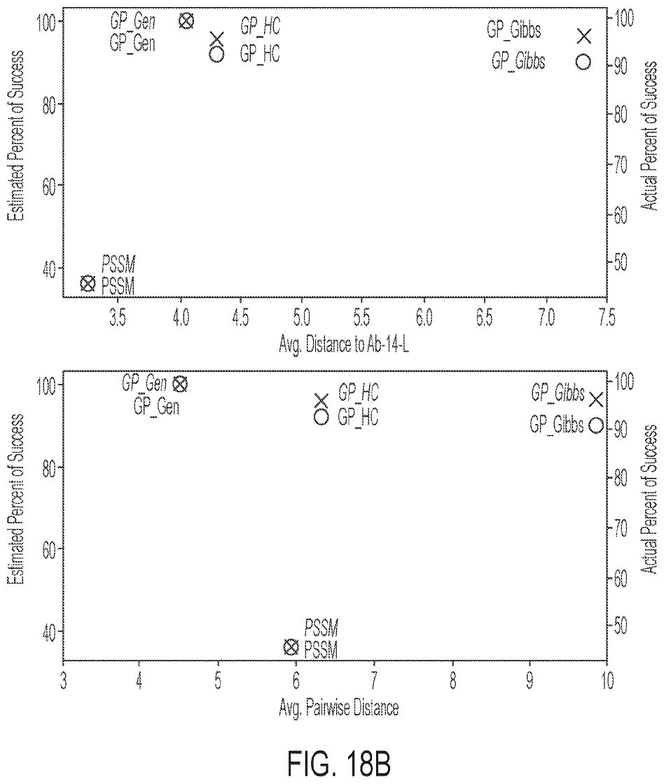

The metric (estimated percent of success) was applied to the designed libraries, and the libraries were ranked. The library rankings were compared based on the estimated and measured percent of success. The rankings are shown in Tables 8-1 and 8-2. For PSSM and ensemble-based libraries, the predicted rankings match well to the actual rankings with a rank correlation of 0.8. For ranking PSSM and GP-based libraries, the metric predicts all rankings correctly for Ab-14-H variant libraries and a rank correlation of 0.8 for Ab-14-L variant libraries. Moreover, as shown in FIGS. 17A-17B and FIGS. 18A-18B, the estimated percent of success captures well the relative performance of designed libraries for both heavy- and light-chain designs. FIG. 17A shows the estimated percent of success determined for ensemble-based libraries for Ab-14-H variants. FIG. 17B shows the estimated percent of success determined for GP-based libraries for Ab-14-H variants. FIG. 18A shows the estimated percent of success determined for ensemble-based libraries for Ab-14-L variants. FIG. 18B shows the estimated percent of success determined for GP-based libraries for Ab-14-L variants.

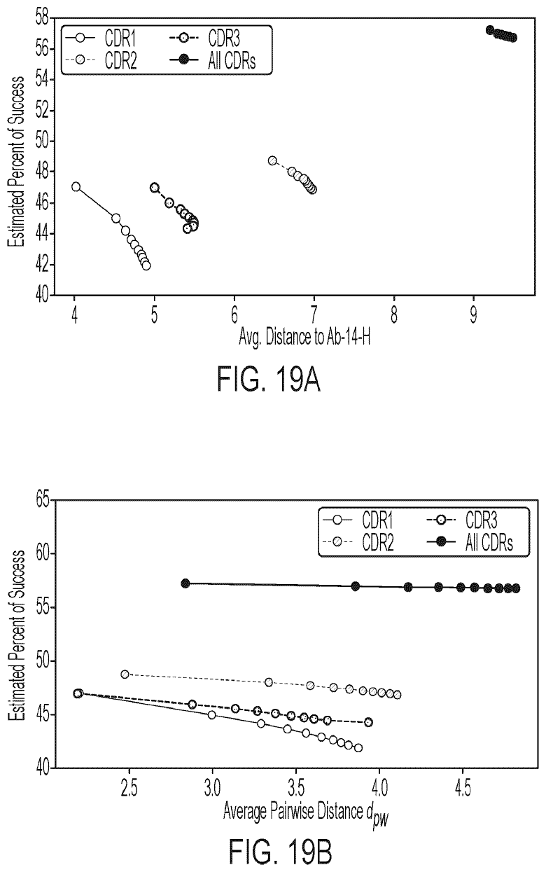

The application of the in silico metric was then extended to comparing the choice of optimizing one CDR to optimizing all three simultaneously. For this comparison, designs were generated using the genetic algorithm sampling over the ensemble-extrapolated fitness landscape. The comparison is shown in FIGS. 19A-19B, which indicate that designing all heavy-chain CDRs leads to sequences with higher estimated percent of success than when designing individual CDRs.

Based on these findings, it is demonstrated that the performance metric can be used to understand design choices and explore tradeoffs between performance and diversity, and to inform library selection and parameter tuning prior to experimental testing.

TABLE 8-1
Ranking of libraries using estimated percent of success.
Ensemble Models

| | Ab-14-H Variant Libraries | | Ab-14-L Variant Libraries | |
|---|---|---|---|---|
| Method | Rank | Rank | Rank | Rank (Actual) |
| PSSM | 4 | 3 | 4 | 4 |
| En-HC | 2 | 2 | 1 | 1 |
| En-GA | 1 | 1 | 2 | 3 |
| En-Gibbs | 3 | 4 | 3 | 2 |

| | |
|---|---|
| Rank Correlation: 0.8 | Rank Correlation: 0.8 |

TABLE 8-2
Ranking of libraries using estimated percent of success.
GP Models

| | Ab-14-H Variant Libraries | | Ab-14-L Variant Libraries | |
|---|---|---|---|---|
| Method | Rank | Rank | Rank | Rank (Actual) |
| PSSM | 4 | 4 | 4 | 4 |
| En-HC | 3 | 3 | 2 | 3 |
| En-GA | 1 | 1 | 1 | 1 |
| En-Gibbs | 2 | 2 | 3 | 2 |

| | |
|---|---|
| Rank Correlation: 1 | Rank Correlation: 0.8 |

### Methods: Training Data for Language Models

Sequences from Pfam and Observed Antibody Space (OAS) databases were used to train four separate language models (i.e., a protein language model, an antibody heavy chain model, an antibody light chain model and a paired heavy-light chain model). The Pfam is a database of curated protein families containing raw sequences of amino acids for individual protein domains. The same data splits as provided in TAPE are used. The train, validation and test splits contain 32,593,668, 1,715,454 and 44,311 sequences, respectively. The full OAS database contains immune repertoires from over 75 studies containing a diverse set of immune states. Studies with naïve human subjects were curated, and redundant sequences were removed across the studies. This results in 37 studies containing 270, 171,931 heavy chain sequences, 9 studies containing 70,838,791 light chain sequences, and 3 studies containing 33,881 heavy-light sequence pairs. The train, validation and test sequences are split based on studies. Given that there are limited heavy-light sequence pairs in the OAS data, to train the paired heavy-light chain model, all the data from OAS heavy chains, OAS light chains and OAS heavy-light sequence pairs was used. For sequence pairs with missing heavy or light chain, the missing chain was left as an empty sequence. Table 9 summarizes the number of sequences in train, validation and test data for the four language model training datasets.

TABLE 9
Train, validation, and test splits for protein/antibody
language model training. Values in parentheses indicate
the number of heavy-light sequence pairs in OAS.

| Datasets | Train | Validation | Test |
|---|---|---|---|
| Pfam | 32,593,668 | 1,715,454 | 44,311 |
| OAS Heavy | 172,524,747 | 47,603,347 | 51,043,837 |
| Chains | | | |
| OAS Light | 70,059,824 | 364,332 | 414,635 |
| Chains | | | |
| OAS Heavy-Light | 242,612,962 | 47,972,437 | 51,459,204 |
| Chain Pairs | (28,391) | (4758) | (732) |

### Methods: Training Language Models

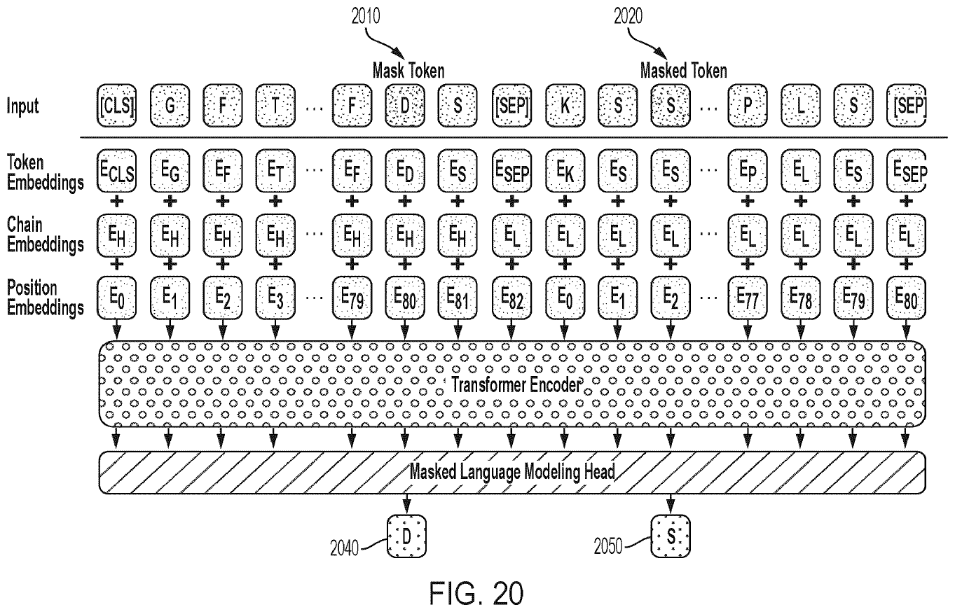

The BERT masked language model was used to encode protein/antibody sequences. FIG. 20 shows an example diagram of masked language modeling with a BERT transformed. The BERT model estimates the probability of an amino acid sequence p(x) by considering the probability distribution over each amino acid at each position conditioned on all other amino acids in the sequence (e.g., Equation 2). Four separate BERT language models were trained, i.e., a protein language model, an antibody heavy chain model, an antibody light chain model and a paired heavy-light chain model, using the Pfam data and OAS data. Specifically, in this example, BERT masked language models were trained with 768 input embedding size, 24 hidden layers, 1024 hidden size, 4096 intermediate feed-forward size and 16 attention heads. All the other architecture details are fixed to their default values used in BERT with Adam optimization. The language model was trained to predict randomly masked amino acids in a single sequence or a sequence pair. In the example, shown in FIG. 20, amino acids 2010 and 2020 of the input sequence are masked, and the language model outputs predictions 2040 and 2050 of those masked amino acids.

For training the protein language model, antibody heavy chain model and antibody light chain model, the input is a single sequence of amino acids. For training the paired heavy-light chain model, the input is a concatenation of heavy and light sequences separated by a special token. Token type IDs are set to 0 for the ‘CLS’ token, 1 for the heavy chain amino acids and 2 for the light chain amino acids to identify two types of chains. Position IDs are set to be the integer position of the amino acid within its respective chain. The Pfam language model was initialized randomly. All other language models were initialized with the pre-trained Pfam model. For all models, the learning rate is set to 10⁻⁵, batch size is 1024 and the warm-up step is 10,000. One training epoch is defined as one full iteration over all the sequences in the training data. All models were trained until convergence of the cross-entropy loss value (which is evaluated on the validation data after every epoch), or until the maximum number of epochs, 10, was reached. All models were implemented in PyTorch and trained on NVIDIA Volta V100 GPUs using a distributed compute architecture.

The standard average perplexity score is used to evaluate the language model performance on the hold-out test data. The perplexity measures how well the trained language models are at predicting the masked tokens. Lower values indicate better performance. The average perplexities of the 4 language models on the respective test data are 13.15 for the Pfam model, 1.56 for the heavy-chain model, 1.43 for the light-chain model and 1.16 for the paired model. When evaluated on the OAS light-chain test data, the average perplexities of the 4 language models are 7.47, 16.40, 1.43 and 1.42, respectively. When evaluated on the OAS heavy-chain test data, the average perplexities of the 4 language models are 12.20, 15.30, 1.56 and 1.56, respectively.

### Methods: Training Sequence-to-Affinity Models

To prepare the training data, the sequences were randomly split in the initial Ab-14-H variant library and Ab-14-L variant library into train/validation/test sets with 0.8/0.0/0.1 split. Since the experimental assay on the initial random scFv library was conducted in triplicate (each scFv sequence has 3 measurements), the average value of all measurements corresponding to the same scFv is used. An assay with an empty measured binding affinity indicates that it is beyond the limit of detection and is deemed a poor binder. Two options were considered for how missing values are treated: dropping the assay with missing value or imputing it with the median value of all assays of the same candidate chain.

Separate target-specific sequence-to-affinity models were trained for Ab-14-H variants and Ab-14-L variants. Model fine-tuning was used as a way to transfer knowledge learned from pre-trained language models to predicting sequence affinities. Two approaches were investigated, which in addition to affinity prediction, provide estimates of prediction uncertainties: an ensemble method and Gaussian Process (GP). Both approaches use learned knowledge from pretrained language models and provide meaningful sequence-to-affinity models from which one can design a diverse antibody library.

The ensemble model includes 16 different trained regression models that were fine-tuned from the 4 pretrained language models with two different regression loss functions and two different data preprocessing steps. The regression models are listed in Table 10. The two loss functions used were the mean squared error (MSE) and the mean absolute error (MAE) between the predicted affinities and measured affinities. For the data preprocessing step, two options were used for treating missing values: dropping the assay with missing value or imputing it with the median value. To train a regression model, the pre-trained BERT language model was fine-tuned (initially trained on massive sequence data without affinity measurements) by adding a linear regression decision head to the BERT model and continuing to train it on a smaller set of scFv sequences with experimental binding measurements. The outputs of the ensemble model are the mean and the standard deviation of the outputs of the 16 regression models.

While the ensemble method may enhance predictive performance, GP is another powerful technique that may be used for quantifying uncertainties. For the GP model, the pretrained heavy-chain language model was used to train the GP model for the heavy chain sequence-to-affinity model and the pretrained light-chain language model was used for the light chain sequence-to-affinity GP model. Sequences were represented by first concatenating the learned vector representations of each amino acid from the pretrained language model, and then performing principal component analysis (PCA) to reduce the vector dimension to 1024. The GP model was trained on these reduced vector representations. Assays with missing values were imputed with the median value in the data preprocessing step. The trained GP model outputs a mean and a standard deviation of the binding affinity prediction.

TABLE 10
Regression models used in the ensemble-based fitness model.

| Name | Base Model | Loss Function | Missing Values |
|---|---|---|---|
| Pfam_drop_l1 | Pfam language | MAE | Drop |
| | model | | |
| Pfam_drop_mse | Pfam language | MSE | Drop |
| | model | | |
| Pfam_median_l1 | Pfam language | MAE | Impute with |
| | model | | median value |
| Pfam_median_mse | Pfam language | MSE | Impute with |
| | model | | median value |
| Heavy_drop_l1 | Heavy-chain | MAE | Drop |
| | model | | |
| Heavy_drop_mse | Heavy-chain | MSE | Drop |
| | model | | |
| Heavy_median_l1 | Heavy-chain | MAE | Impute with |
| | model | | median value |
| Heavy_median_mse | Heavy-chain | MSE | Impute with |
| | model | | median value |
| Light_drop_l1 | Light-chain | MAE | Drop |
| | model | | |
| Light_drop_mse | Light-chain | MSE | Drop |
| | model | | |
| Light_median_l1 | Light-chain | MAE | Impute with |
| | model | | median value |
| Light_median_mse | Light-chain | MSE | Impute with |
| | model | | median value |
| Paired_drop_l1 | Paired model | MAE | Drop |
| Paired_drop_mse | Paired model | MSE | Drop |
| Paired_median_l1 | Paired model | MAE | Impute with |
| | | | median value |
| Paired_median_mse | Paired model | MSE | Impute with |
| | | | median value |

### Methods: ML Extrapolated Fitness Functions

To generate a high affinity scFv library in silico, a Bayesian-based acquisition function extrapolated from the sequence-to-affinity model was used to construct the scFv fitness landscape. In contrast to non-Bayesian settings where the sequence is mapped directly to estimated affinity, the fitness function is defined to be a mapping from the entire scFv sequence to a posterior probability that the estimated binding affinity aff(x) of the sequence x is better than the threshold σ (e.g., Equation 1). The threshold was set to the averaged assayed value of Ab-14 in the training data. Assuming a Gaussian distribution, f(x) can be computed using the mean and standard deviation of the prediction from the trained sequence-to-affinity model. For each scFv chain (Ab-14-H and Ab-14-L), two fitness functions were computed. The two fitness functions were extrapolated from the ensemble model and GP model, respectively. The proposed fitness function captures the model uncertainty during the optimization and enables us to estimate the performance of the antibody designs prior to experimental testing.

### Methods: Optimization Strategies

The goal of this example was to sample scFv sequences with the highest extrapolated fitness value f(x). The optimization was performed using 3 different sampling algorithms: a greedy algorithm called hill climb (HC), an evolutionary algorithm called genetic algorithm (GA) and Gibbs sampling. The HC and GA sampling processes were initialized using the 10 strongest binders (seed sequences) from the supervised training data and the Gibbs sampling using the strongest binders from the training data.

For the hill climb algorithm, the optimization was initialized by randomly mutating a seed sequence with an expected number of k=2 mutations. At each step, the algorithm performs a local search around the current sequence and samples the next sequence that has the highest fitness value. The search continues until it can no longer find a sequence that has a better fitness value than the current sequence. The local search space was defined to be the 1000 mutants of the current sequence, consisting of all the k=1 mutations and random k=2 mutations. The greedy-based hill climb was run 100 times with random restart around a random seed sequence.

The genetic algorithm (GA) is an evolution-based search heuristic, where the fittest individuals are selected to produce offspring of the next generation. The population was initialized with a random seed sequence from the top 10 binders. Parents were chosen from the current population based on the Wright-Fisher model of evolution where members of the current population become parents with a probability exponential to their fitness values, that is, p(x)˜exp (f(x)/β). Sequences with high fitness have more chances to pass their genes to the next generation. A single-point crossover was performed on two parent sequences randomly selected from the parent population and followed by randomly mutating individual child sequences with an expected k=1 mutation. The algorithm was terminated when it no longer produced new sequences (the population converged). The algorithm was run 100 times; each was initialized from a random seed sequence. The parameter β was set to be 0.2 for the ensemble-based fitness function and 0.5 for the GP-based fitness function. The selection of parameter value β directly affects the diversity of generated sequence designs. Depending on the design needs, this parameter can be tuned to adjust the overall library diversity. Due to limited understanding of the extrapolation power of ML models at the time of sequence design, the β parameter was manually selected around its default value used in FLEXS.

Gibbs sampling is a Markov Chain Monte Carlo (MCMC) algorithm that samples a sequence according to some joint distribution by generating random variates from each of the full conditional distributions. The algorithm was initialized from the top seed sequence (the sequence with the strongest binding affinity in the training data). At each step, a position i in the sequence was randomly selected, a mutant [symbol] was sampled at the selected position with a conditional probability,

$$p ( {x}_{i} \vert {x}_{1} , … {x}_{i - 1} , {x}_{i + 1} , … {x}_{L} ) \qquad ( Equation 3 )$$

and the sequence was updated by replacing the i^{th} token with the sample token [symbol] . The conditional probability was defined to be exponential to the fitness value, that is,

$$p ( {x}_{i} \vert {x}_{1} , … {x}_{i - 1} , {x}_{i + 1} , … {x}_{L} ) ∼ exp ( γ * f ( x ) ) \qquad ( Equation 4 )$$

The Gibbs sampling was run once with 30,000 iterations. The value γ was set to be 18 for the Ab-14-H ensemble-based fitness function, and 20 for both the Ab-14-L ensemble- and GP-based fitness function. Multiple γ values were used to sample the Ab-14-H GP-based fitness function. This is due to the limited number of sequences that can be sampled at any specific γ value for the given fitness function. To ensure that enough sequences can be sampled, γ=10, 3, 2 was used, and the Gibbs algorithm was run three times to sample a sufficient number of sequences.

### Methods: ML-Optimized ScFv Libraries

For each scFv chain (Ab-14-H variants and Ab-14-L variants), two fitness functions extrapolated from the ensemble and GP model, respectively, were constructed. For each fitness function, optimization was performed using three sampling strategies. This resulted in 6 libraries per chain: 3 libraries from optimizing the ensemble-based fitness function (namely, En-HC, En-GA and En-Gibbs), and 3 libraries from optimizing the GP-based fitness function (namely, GP-HC, GP-GA, GP-Gibbs). The generated sequences were rank-ordered based on their fitness score per library and the top 6000 sequences were selected per library for experimental validation. FIGS. 8A-8B and 9A-9B show the distribution of the designed sequences with respect to various mutational distances to demonstrate the library diversity: (1) mutational distance to the candidate scFv Ab-14, and (2) pairwise mutational distance in a library. The first distance metric measures the number of mutations the designed antibodies are from Ab-14. The second distance metric measures the intra-library diversity.

### Methods: Evolution Directed Libraries

Two baseline libraries were built based on conventional directed evolution strategies: random mutations and the PSSM-based method. The random mutation library was constructed by randomly mutating amino acid tokens from the seed sequences in the training data with a k=2 average number of mutations. Using this method, 2097 Ab-14-H heavy-chain variants and 477 Ab-14-L light-chain variants were generated for experimental testing.

For the PSSM-based library, sequences in the training data with measured affinities that are as good or better than the candidate scFv Ab-14 were used. The PSSM was fitted by counting the occurrence of each amino acid at each position in the CDRs with a small pseudocount. The fitted PSSM is a matrix of probability scores for each amino acid at each position, representing the statistical patterns of the training sequences that are better than Ab-14. Samples were drawn to generate designs based on the fitted PSSM. Contrary to the random mutation approach, the PSSM-based approach is not restricted to a pre-defined mutational distance and could generate sequences that are potentially far from the candidate antibody if the computed PSSM allows. The PSSM method resulted in 7748 Ab-14-H heavy-chain variant designs and 8257 Ab-14-L light-chain variant designs that were sent for experimental testing. FIGS. 10C and 10F show the distribution of the generated sequences with respect to the mutational distances.

### Methods: Experimental Validation of Designed Sequences

An engineered yeast mating assay was used to empirically measure the relative binding strength of the ML-designed sequences. Yeast peptone dextrose (YPD), yeast peptone galactose (YPG), and synthetic drop out (SDO) media supplemented with 80 mg/mL adenine were made according to standard protocols. Suppliers used for the yeast media are as follows: Bacto Yeast Extract (Life Technologies), Bacto Tryptone (Fisher BioReagents), Dextrose (Fisher Chemical), Galactose (Sigma-Aldrich), Adenine (ACROS Organics), Yeast Nitrogen Base w/o Amino Acids (Thermo Scientific), SC-His-Leu-Lys-Trp-Ura Powder (Sunrise Science Products), Yeast Synthetic Drop-out Medium Supplements (Sigma-Aldrich), L-Histidine (Fisher BioReagents), L-Tryptophan (Fisher BioReagents), L-Leucine (Fisher BioReagents), Uracil (ACROS Organics), and Bacto Agar (Fisher BioReagents).

AlphaSeq compatible plasmids encoding yeast surface display cassettes were constructed by Twist Bioscience and resuspended at 100 ng/μL in molecular grade water (Corning). 100 ng of plasmid was digested with Pmel enzyme (NEB) for 1 hr at 37° C. to linearize, leaving chromosomal homology for integration into the ARS314 locus at both 5′ and 3′ ends. Yeast transformations were performed with Frozen-EZ Yeast Transformation Kit II (Zymo Research) according to manufactures instructions. Yeast were plated on SDO-Trp plates and grown at 30° C. for 2-3 days. Successful transformants were struck out onto YPAD plates and grown overnight at 30° C.

To validate protein expression, yeast were inoculated in YPAD and grown overnight at 30° C. Yeast were labelled with FITC-anti-C-myc antibody (Immunology Consultants Laboratory, Inc.) in PBS (Gibco)+0.2% BSA (Thermo Fisher Scientific) for 30 minutes at RT. Yeast were pelleted and resuspended in PBS+0.2% BSA and read on a LSRII cytometer.

To construct the DNA library, a 300 bp oligonucleotide pool synthesized by Twist Bioscience was resuspended at 20 ng/μL in molecular grade water (Corning). Libraries were PCR amplified from the oligonucleotide pool using KAPA DNA polymerase (Roche). The oligonucleotide amplification fragment was inserted into the seed scFv backbone using Gibson isothermal assembly (NEB), as well as a second DNA fragment containing a randomized DNA barcode. The assembled barcoded antibody DNA library was PCR amplified. Fragments were run on a 0.8% agarose gel and extracted using Monarch Gel Purification kit (NEB).

For the yeast library transformation, MATa AlphaSeq yeast were grown for 16 hours in YPAG media to induce SceI expression. All spin steps were performed at 3000 RPM for 5 minutes. Yeast were spun down and washed once in 50 mL 1 M Sorbitol (Teknova)+1 mM CaCl2 (Sigma-Aldrich) solution. Washed yeast were resuspended in a solution of 0.1 M LiOAc (ACROS Organics)/1 mM DTT (Roche) and incubated shaking at 30° C. for 30 minutes. After 30 minutes, yeast were spun down and washed once in 50 mL 1 M Sorbitol+1 mM CaCl2 solution. Yeast were resuspended to a final volume of 400 μL in 1 M Sorbitol+1 mM CaCl2 solution and incubated with DNA for at least 5 minutes on ice. Yeast were electroporated at 2.5 kV and 25 uF (BioRad). Immediately following electroporation, yeast were resuspended in 5 mL of 1:1 solution of 1 M Sorbitol:YPAD and incubated shaking at 30° C. for 30 minutes. Recovered yeast cells were spun down and resuspended in 50 mL of SDO-Trp media and transferred to a 250 mL baffled flask. 20 μL of resuspended cells were plated on SDO-Trp to determine transformation efficiency. Both the flask and plate were incubated at 30° C. for 2-3 days. After 2-3 days, transformation efficiency was determined by counting colonies on the SDO-Trp plate.

For nanopore barcode mapping, genomic DNA from yeast libraries was extracted using Yeast DNA Extraction Kit (Thermo Fisher Scientific) following the manufacturer's instructions. A single round of qPCR was performed to amplify a fragment pool from the genomic DNA containing the gene through the associated DNA barcode. qPCR was terminated before saturation to minimize PCR bias, generally between 15-20 cycles. The final amplified fragment was concentrated with KAPA beads (Roche), quantified with a Quantus (Promega), prepped with an SQK-LSK-110 ligation kit (Oxford Nanopore) and sequenced with a Minion R10 flow cell (Oxford Nanopore) following the manufacturer's instructions. Each sequencing read was aligned to the set of expected antibody sequences from the in silico antibody library using minimap2 to determine the mapping between DNA barcodes and antibody sequence; only DNA barcodes with at least 2 reads observed were considered, and each DNA barcode was matched to the most common minimap2 antibody match among its constituent reads.

Library-on-library AlphaSeq assays were performed. Two mL of saturated MATa and MATalpha library were combined in 800 mL of YPAD media and incubated at 30° C. in a shaking incubator. Six technical replicates were performed. After 16 hr, 100 mL of yeast culture was washed once in 50 mL of sterile molecular grade water (Corning) and transferred to 600 mL of SDO-lys-leu with 100 nM ß-estradiol (Sigma-Aldrich) for 24 hr. After 24 hr, 100 mL of yeast was transferred to fresh SDO-lys-leu with 100 nM ß-estradiol for an additional 24 hr. In addition to the antibody libraries described above, control yeast strains comprising a small network of BCL2-family proteins were included in each experiment to act as a set of standards for which BLI-derived interaction affinities were known a priori.

To prepare the library for next-generation sequencing, genomic DNA was extracted using Yeast DNA Extraction Kit (Thermo Fisher Scientific) following manufacturer's instructions. qPCR was performed to amplify a fragment pool from the genomic DNA and to add standard Illumina sequencing adaptors and assay specific index barcodes. qPCR was terminated before saturation to minimize PCR bias, generally between 23-27 cycles. The final amplified fragment was concentrated with KAPA beads (Roche), quantified with a Quantus (Promega), and sequenced with a NextSeq 500 sequencer (Illumina).

Sequencing data were analyzed to identify the MATa and MATalpha barcode pairs present among diploid yeast. The observed number of sequencing reads for each MATa/MATalpha combination were normalized according to frequency among haploid yeast to account for uneven distribution of the input populations. Each au pair was then assigned a score representing the ratio of observed sequencing reads to expected sequencing reads assuming random mating. A linear regression was performed comparing these normalized sequencing scores to known affinities for the control yeast strains and this regression was utilized to assign estimated affinities to all other au pairs for each mating replicate.

Tables 3 and 4 summarize the number and percentage of sequences present in the experimental data for Ab-14-H and Ab-14-L designs, respectively. All generated data with experimental affinity measurements are made publicly available for research use. To use the experimentally collected affinity data for evaluating the performance of designed scFv sequences, designs that are present in the experimental data were used. For sequences that are present in the affinity data and have at least three out of six empirical affinity values, the values are averaged and used as ground-truth measured affinities. Sequences with two or fewer empirical measurements are considered poor binders and are included in the performance evaluation as un-successful designs.

Methods: T-Distributed Stochastic Neighbor Embedding (t-SNE)

tSNE is used to visualize high-dimensional scFv sequences while approximately preserving the edit distance between sequences. Specifically, all scFv sequences were encoded using a one-hot encoder; for any pair of one-hot encoded scFv sequences, the L1-norm between them equals the edit distance. Then the t-SNE dimensionality reduction is applied to project one-hot encoded sequences into a 2-D space as shown in FIGS. 14A-14B. Python scikit-learn package was used to perform t-SNE with the L1-norm and PCA initialization43. For Ab-14-H variants, the perplexity and learning rate are set to be 500 and 200, respectively. For Ab-14-L variants, the perplexity and learning rate are set to be 500 and 500, respectively.

### Methods: Biophysical Property Calculation, Statistical Analysis of Libraries

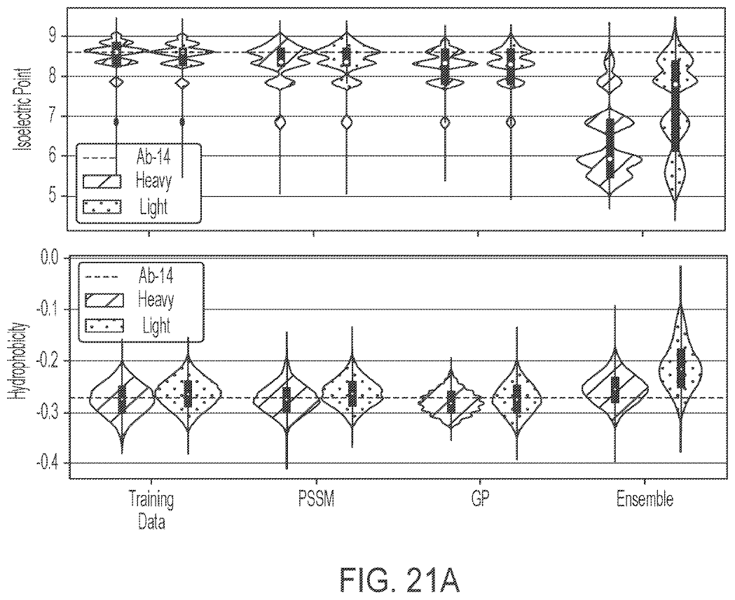

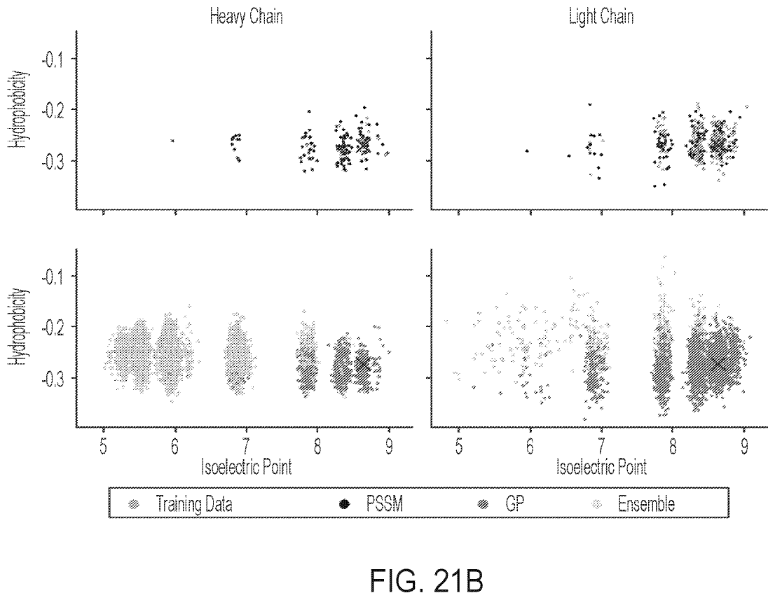

For the biophysical property analysis of designed libraries, isoelectric points and hydrophobicity, which are physicochemical descriptors known to influence the solution behavior of antibodies, were computed. These properties were calculated based on the sequences of the heavy and light chain variants in each library using BioPython. Specifically, for the heavy chain, each heavy-chain design was concatenated with the fixed light-chain sequence; for the light chain, the fixed heavy-chain sequence was concatenated with each light-chain design. Isoelectric points were calculated using pK values. Hydrophobicity was calculated using the Kyte & Doolittle index. The hydrophobicity score of each amino acid was averaged over the sequence of each variant to give an overall hydrophobicity score for each sequence. FIGS. 21A-21B show the distribution of isoelectric and hydrophobicity. In particular, FIG. 21A is a violin plot of isoelectric points (pI) and hydrophilicities of heavy and light chain sequences in the training data, and PSSM-, GP-, and ensemble-designed libraries (center: median; limits: 1st and 3rd quartile; whiskers: +/−1.5 IQR). Evaluations were performed over n=26454, 6510, 12407, 14835 Ab-14-H variants and n=26224, 8188, 17500, 17872 Ab-14-L variants from the Training data, PSSM-, GP-, and ensemble-generated libraries, respectively. The dashed lines represent the value of the corresponding candidate Ab-14. The pI values calculated for most of the Ab-14-H and Ab-14-L variants are in the 7.5-9.0 interval. The exception is in the ensemble-based method, in which it exhibits a wider pI value range (5.0-9.0), and many Ab-14-H variants have acidic pI (below 6.5). Similarly, the hydrophobicity values of ensemble-based libraries also have a wider value range. FIG. 21B shows scatter plots showing the joint distribution of pI and hydrophilicities for sequences with strong binding affinity (measured binding affinity <=1 nM). The top row shows the results for the training data and PSSM libraries; the bottom row shows the GP and ensemble libraries. The ‘x’ marker indicates isoelectric point and hydrophobicity of Ab-14. The designed strong binders cover a wide range of these biophysical properties.

### Computer Implementation

An illustrative implementation of a computer system 2200 that may be used in connection with any of the embodiments of the technology described herein (e.g., such as process 300 of FIG. 3 and process 400 of FIG. 4) is shown in FIG. 22. The computer system 2200 includes one or more processors 2210 and one or more articles of manufacture that comprise non-transitory computer-readable storage media (e.g., memory 2220 and one or more non-volatile storage media 2230). The processor 2210 may control writing data to and reading data from the memory 2220 and the non-volatile storage device 2230 in any suitable manner, as the aspects of the technology described herein are not limited to any particular techniques for writing or reading data. To perform any of the functionality described herein, the processor 2210 may execute one or more processor-executable instructions stored in one or more non-transitory computer-readable storage media (e.g., the memory 2220), which may serve as non-transitory computer-readable storage media storing processor-executable instructions for execution by the processor 2210.

Computing device 2200 may include a network input/output (I/O) interface 2240 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing device 2200 may also include one or more user I/O interfaces 2250, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

1. A method for designing antibodies for binding to a target, the method comprising:
obtaining an amino acid sequence of a candidate antibody wherein the candidate antibody binds to the target with a candidate binding affinity;
determining, for antibodies in a set of antibodies, probabilities that binding affinities between the antibodies and the target are greater than the candidate binding affinity, the antibodies in the set of antibodies having different amino acid sequences, the antibodies including a first antibody having a first amino acid sequence, and the probabilities including a first probability that a first binding affinity between the first antibody and the target is greater than the candidate binding affinity, wherein determining the first probability comprises:
processing the first amino acid sequence of the first antibody using a trained machine learning model to obtain a first output indicative of the first binding affinity between the first antibody and the target; and
determining the first probability using the first output indicative of the first binding affinity between the first antibody and the target; and
identifying a subset of the set of antibodies based on the determined probabilities that the binding affinities are greater than the candidate binding affinity.

2. (canceled)

3. The method of claim 1,
wherein determining the probabilities that the binding affinities between the antibodies and the target are greater than the candidate binding affinity further comprises:
determining a second amino acid sequence of a second antibody in the set of antibodies based on (i) the first probability that the first binding affinity is greater than the candidate binding affinity and (ii) the first amino acid sequence of the first antibody.

4. The method of claim 3, further comprising:
after determining the second amino acid sequence, determining a second probability that a second binding affinity between the second antibody and the target is greater than the candidate binding affinity.

5. (canceled)

6. (canceled)

7. The method of claim 1, further comprising:
identifying the first antibody from among a training set of antibodies having known binding affinities.

8. The method of claim 1, wherein identifying the subset of the set of antibodies comprises:
ranking the antibodies in the set of antibodies by the probabilities determined for the antibodies; and
identifying the subset of the set of antibodies based on the ranking.

9. The method of claim 8,
wherein ranking the antibodies in set of antibodies by the probabilities determined for the antibodies comprises ranking the antibodies from a highest probability of the determined probabilities to a lowest probability of the determined probabilities.

10. The method of claim 8,
wherein identifying the subset of the set of antibodies based on the ranking comprises identifying a predetermined number of antibodies associated with highest probabilities of the determined probabilities.

11. The method of claim 1, further comprising:
identifying, from among the identified subset of the set of antibodies, one or more antibodies having at least one pre-determined property; and
producing the identified one or more antibodies having the at least one pre-determined property.

12. The method of claim 1, wherein the first output indicative of the first binding affinity between the first antibody and the target comprises a mean of the first binding affinity and a standard deviation of the first binding affinity.

13. (canceled)

14. The method of claim 1, wherein the trained machine learning model comprises at least one regression model.

15. The method of claim 14, wherein the at least one regression model is trained to predict, for an amino acid sequence of an antibody, a binding affinity between the antibody and the target.

16. (canceled)

17. The method of claim 1, wherein the trained machine learning model comprises a Gaussian Process model.

18. (canceled)

19. The method of claim 1, wherein the trained machine learning model comprises at least one language model trained to encode amino acid sequences.

20. The method of claim 19, wherein the at least one language model is trained to predict masked amino acids in at least one amino acid sequence.

21. The method of claim 19, wherein the trained machine learning model further comprises:
at least one regression model fine-tuned from the at least one language model, or a probabilistic model fine-tuned from the at least one language model.

22. The method of claim 21, wherein processing the first amino acid sequence of the first antibody using the trained machine learning model comprises:
processing the first amino acid sequence using the at least one language model to obtain an encoded amino acid sequence, and
processing the encoded amino acid sequence using the at least one regression model or the at least one probabilistic model to obtain the first output indicative of the first binding affinity between the first antibody and the target.

23. (canceled)

24. (canceled)

25. A system, comprising:
at least one computer hardware processor; and
at least one non-transitory computer-readable storage medium storing processor-executable instructions that, when executed by the at least one computer hardware processor, cause the at least one computer hardware processor to perform a method for designing antibodies for binding to a target, the method comprising:
obtaining an amino acid sequence of a candidate antibody wherein the candidate antibody binds to the target with a candidate binding affinity;
determining, for antibodies in a set of antibodies, probabilities that binding affinities between the antibodies and the target are greater than the candidate binding affinity, the antibodies in the set of antibodies having different amino acid sequences, the antibodies including a first antibody having a first amino acid sequence, and the probabilities including a first probability that a first binding affinity between the first antibody and the target is greater than the candidate binding affinity, wherein determining the first probability comprises:
processing the first amino acid sequence of the first antibody using a trained machine learning model to obtain a first output indicative of the first binding affinity between the first antibody and the target; and
determining the first probability using the first output indicative of the first binding affinity between the first antibody and the target; and
identifying a subset of the set of antibodies based on the determined probabilities that the binding affinities are greater than the candidate binding affinity.

26. (canceled)

27. A method of training a machine learning model to predict binding affinities between antibodies and a target, the method comprising:
using at least one computer hardware processor to perform:
training at least one language model to encode amino acid sequences;
obtaining training data using a candidate amino acid sequence of a candidate antibody, wherein the candidate antibody binds to the target with a candidate binding affinity; and
training the machine learning model to predict the binding affinities between the antibodies and the target using the at least one trained language model and the obtained training data.

28. The method of claim 27, wherein training the at least one language model to encode the amino acid sequences comprises training the at least one language model to predict masked amino acids in at least one amino acid sequence.

29. The method of claim 27, wherein training the at least one language model comprises training a protein language model using protein training data, the protein training data comprising amino acid sequences for individual protein domains.

30.-47. (canceled)
